(19) United States
(12) Patent Application Publication
(10) Pub. No.: US 2006/0172415 A1
(43) Pub. Date: Aug. 3, 2006

(54) Oligodendrocyte precursor cells and method of obtaining and culturing the same

(76) Inventor: Hiroshi Okazaki, Potomac, MD (US)

Correspondence Address: SUGHRUE MION, PLLC, 2100 PENNSYLVANIA AVENUE, N.W., SUITE 800, WASHINGTON, DC, 20037, US

(21) Appl. No.: 10/564,819

(22) PCT Filed:
(86) PCT No.: PCT/IB04/02670

Related U.S. Application Data
(60) Provisional application No. 60/487,933, filed on Jul. 18, 2003.

(51) Int. Cl. C12N 5/08

(52) U.S. Cl. 435/368

(57) ABSTRACT

The invention describes a self-renewing, phenotypically homogeneous population of oligodendrocyte precursor cells having a synchronized developmental stage and methods of obtaining a self-renewing phenotypically homogeneous population of oligodendrocyte precursor cells. Other methods include methods of maintaining and storing a homogeneous population of oligodendrocyte precursor cells for a prolonged period of time without change in the characteristics of the cells and methods of dedifferentiating oligodendrocyte precursor cells. The self-renewing, phenotypically homogeneous population of oligodendrocyte precursor cells or homogeneous population of oligodendrocytes may be useful for treating a patient having a CNS disorder or condition.

### RELATED APPLICATIONS

The present application claims benefit of U.S. Provisional Application No. 60/487,933, filed Jul. 18, 2003.

### FIELD OF THE INVENTION

The present invention relates to methods of obtaining a self-renewing, phenotypically homogeneous population of oligodendrocyte precursor cells having a synchronized developmental stage, and to the cells obtained by the methods of the present invention.

The present invention further relates to methods of maintaining a self-renewing, phenotypically homogeneous population of oligodendrocyte precursor cells having a synchronized developmental stage for a prolonged period of time without change in the characteristics of the cells.

The oligodendrocyte precursor cells of the invention can be dedifferentiated back to an earlier developmental stage by sequentially utilizing cell dissociation (through a digestive reagent, such as a trypsin), followed by defined medium conditions. Thus, the present invention also relates to a method of obtaining a differentiated and dedifferentiated homogeneous population of oligodendrocyte precursor cells or oligodendrocytes having a synchronized developmental stage.

Finally, the present invention relates to a method of treating a patient using the cells of the present invention and to a method of screening drug candidates for use in treatment of demyelinating and neuronal degenerative diseases that result in the reduction of myelin.

### BACKGROUND OF THE INVENTION

The axons of many vertebrate neurons are insulated by a myelin sheath, which greatly increases the rate at which an axon can conduct an action potential. Oligodendrocytes are responsible for the formation of myelin in the central nervous system. These oligodendrocytes wrap layer upon layer of their own plasma membrane in a tight spiral around the axon to form a sheath, thereby insulating the axonal membrane so that almost no current leaks across it. The sheath is interrupted at regularly spaced nodes of Ranvier, where almost all the sodium channels in the axon are concentrated. Because the ensheathed portions of the axon membrane have excellent cable properties, a depolarization of the membrane at one node almost immediately spreads passively to the next node. Thus, an action potential propagates along a myelinated axon by jumping from node to node. This type of conduction has two main advantages: action potentials travel faster, and metabolic energy is conserved because the active excitation is confined to the small regions of axonal plasma membrane at nodes of Ranvier.

The importance of myelination is dramatically demonstrated by the demyelinating disease multiple sclerosis, in which myelin sheaths in some regions of the central nervous system are destroyed by an unknown mechanism. The importance of myelination is also strongly demonstrated in many neurodegenerative disease, in which myelinated neurons are injured. Where this happens, the propagation of nerve impulses is greatly slowed, often with devastating neurological consequences.

Oligodendrocytes appear to be terminally differentiated cells which do not undergo further cell division in vivo, and therefore are difficult to culture in vitro long term (Verity et al., J. Neurochem., 60:577, 1993). Oligodendrocyte precursor cells, which have proliferative capacity and differentiation potential, offer a system by which cellular and molecular mechanisms of cell differentiation and myelination/demyelination/remyelination may be studied in vitro and provide a source for promoting myelinatlon/remyelination in vivo. However, oligodendrocytes develop asynchronously from oligodendrocyte precursor cells in the central nervous system (CNS) and so it is also considered that they might be a phenotypically heterogeneous population (Skoff et al., J. Comp. Neurol. 169:313-334, 1976). Indeed, cultures initiated from dissociated perinatal brain are an Inherently heterogeneous population with unsynchronized, developmental maturity. It has therefore been difficult to isolate phenotypically homogeneous populations of primary oligodendrocyte precursor cells having the same developmental stage.

Thus, there remains a need in the art for a self-renewing, phenotypically homogeneous population of synchronous oligodendrocyte precursor cells having a synchronized developmental stage and a method for obtaining, maintaining, and storing the same.

### SUMMARY OF THE INVENTION

In one aspect, the present invention provides a method for obtaining a self-renewing, phenotypically homogeneous population of oligodendrocyte precursor cells having a synchronized developmental stage. The method comprises culturing a heterogeneous population of oligodendrocyte precursor cells having an unsynchronized developmental stage in a medium comprising an effective amount of a fibroblast growth factor (FGF), preferably basic FGF (bFGF), and in the substantial absence of platelet-derived growth factor (PDGF). The method yields a self-renewing, phenotypically homogeneous population of oligodendrocyte precursor cells having a synchronized developmental stage which may be characterized by one or more of the following abilities: (1) self-renewing proliferation in response to bFGF without differentiating, (2) terminal differentiation into a homogeneous population of oligodendrocytes in the absence of mitogens or serum, (3) generation of a homogeneous population of type 2 astrocytes in the presence of BMP-2, (4) dedifferentiation, (5) promotion of myelination in vitro and in vivo, (6) lack of potential to differentiate into type 1 astrocytes, and (7) a high degree of survival without change in the characteristics of the cells upon thawing after being frozen.

Changes in the characteristics of the cells are determined based on the specific characterization of these cells, such as an ability of self-renewing proliferation, the same as multi-potent stem cells, and the ability of a phenotypically homogeneous population of oligodendrocyte precursor cells having a synchronized developmental stage to be stored with high survival upon recovery and without change in the characteristics of the cells, based the specific characterization of these cells, which includes high tolerance to freeze-thaw treatments.

The present invention also includes self-renewing, phenotypically homogeneous populations of oligodendrocyte precursor cells having a synchronized developmental stage that can be restricted to a single differentiation lineage. For example, such cells can be limited in that the entire population differentiates into a single lineage, such as into mature oligodendrocytes or into type 2 astrocytes.

In another aspect of the invention, the self-renewing, phenotypically homogeneous population of oligodendrocyte precursor cells having a synchronized developmental stage may also be maintained indefinitely in culture. The present invention thus also provides a method for obtaining, maintaining, and storing indefinitely in culture a self-renewing, phenotypically homogeneous population of oligodendrocyte precursor cells having a synchronized developmental stage, comprising culturing the homogeneous population of oligodendrocyte precursor cells having a synchronized developmental stage in a medium comprising an effective amount of a FGF, preferably bFGF, and in the substantial absence of PDGF.

The present invention also provides a method of storing viable, frozen, undifferentiated, and homogeneous oligodendrocyte precursor cells by freezing the oligodendrocyte precursor cells in freezing medium with or without mitogens. Upon thawing, the oligodendrocyte precursor cells are recovered with a high degree of survival and retain the same phenotypic and developmental characteristics they possessed before they were frozen.

The oligodendrocyte precursor cells obtained by the methods of the present invention can be used to generate homogeneous and synchronous populations of mature oligodendrocytes in the absence of mitogens or serum, and have the ability to myelinate neuronal axons. The oligodendrocyte precursor cells obtained by the methods of the present invention can also be used to generate homogeneous populations of type 2 astrocytes, lacking in the ability to proliferate in the presence of specific mitogens, such as bone morphogenic protein 2 (BMP-2) and BMP-4. The oligodendrocyte precursor cells of the present invention further do not generate type 1 astrocytes.

The present invention further provides a method of obtaining a homogeneous population of dedifferentiated oligodendrocyte precursor cells of a developmentally or phenotypically earlier stage than the initially isolated oligodendrocyte precursor cells. The method comprises culturing the oligodendrocyte precursor cell, or a homogeneous population of oligodendrocyte precursor cells having a synchronized developmental stage, In a medium comprising at least one factor that promotes dedifferentiation. The factor that promotes dedifferentiation may be one or more of bFGF, PDGF, NT-3 or other growth factors. The dedifferentiated oligodendrocyte precursor cell (or a homogeneous population of dedifferentiated oligodendrocyte precursor cells having a synchronized developmental stage) may be capable of re-differentiating into oligodendrocytes and type 2 astrocytes.

The present invention further provides a method of obtaining a self-renewing, phenotypically homogeneous population of proliferating oligodendrocyte precursor cells of a developmentally or phenotypically later stage than the initially isolated oligodendrocyte precursor cells. The method comprises culturing the oligodendrocyte precursor cell, or a homogeneous population of oligodendrocyte precursor cells having a synchronized developmental stage, in a medium comprising at least one factor that promotes development of a more differentiated stage. The more differentiated stage is further characterized by the ability of the cells in the more differentiated stage to proliferate. The factor that promotes a more differentiated, proliferating stage may be a lower dosage of bFGF or other growth factors.

The self-renewing, phenotypically homogeneous population of oligodendrocyte precursor cells having a synchronized developmental stage of the present invention provides a system for screening compounds that affect the biological function and/or differentiation state of oligodendrocyte precursor cells. Thus, the present invention further provides a method of screening for compounds, the method comprising contacting the self-renewing, phenotypically homogeneous population of oligodendrocyte precursor cells having a synchronized developmental stage with a test compound, and detecting a change in the oligodendrocyte precursor cells and/or In the culturing medium. The change may be an Increase or reduction in any characteristic of the oligodendrocyte precursor cell and/or in levels of any materials in the culturing medium. The characteristic may be, for example, one or more of a change in: myelination, differentiation into oligodendrocytes or type 2 astrocytes, proliferation speed, cell migration, viability, gene expression, protein expression, protein levels in the culturing medium, dedifferentiation, or cell morphology.

The self-renewing, phenotypically homogeneous population of oligodendrocyte precursor cells having a synchronized developmental stage of the invention is also useful for treating a patient, such as in cell therapy. A patient may be suffering or have a condition of the nervous system that results from the deterioration of, or damage to, myelin sheathing. The present invention provides a method of treating a patient comprising administering to the patient a therapeutically effective amount of the oligodendrocyte precursor cell of the invention. In an embodiment of the invention, the oligodendrocyte precursor cell may contain a nucleic acid vector or biological vector that directs the expression of a desired gene(s) In the patient.

### BRIEF DESCRIPTION OF THE DRAWINGS

FIG. 1 is a schematic representation of the various cell types in the central nervous system (CNS) characterized by developmental markers and cell morphology.

FIG. 2 is a phase-contrast photograph of a homogeneous and developmentally synchronized population of rat oligodendrocyte precursor cells.

FIG. 3 is a phase-contrast photograph of a homogeneous and developmentally synchronized population of human oligodendrocyte precursor cells.

FIG. 4 shows phase-contrast photographs and fluorescent images of O4(+)O1(+) oligodendrocyte precursor cells immunocytochemically stained with Cy3-conjugated anti-O4 antibody or with Cy3-conjugated anti-O1 antibody.

FIGS. 5A-5C are photographs of oligodendrocytes. FIGS. 5A and 5B are phase-contrast photographs of rat and human oligodendrocytes, respectively.

FIG. 5C shows a human oligodendrocyte in phase contrast and the same oligodendrocyte double-stained with Cy3-conjugated anti-O1 antibody and FITC-conjugated anti-MBP antibody.

FIGS. 6A and 6B are photographs of rat oligodendrocyte precursor cells differentiated into type 2 astrocytes. FIG. 6A is a phase-contrast photograph of the type 2 astrocytes and FIG. 6B is a fluorescent image of the same cells showing expression of the glial fibrillary acidic protein (GFAP).

FIGS. 7A-7D are photographs of cells that arose from O4(+)O1(+) oligodendrocyte precursor cells. FIG. 7A shows a phase-contrast photograph of O4(+)O1(+) oligodendrocyte precursor cells and a fluorescent image of the cells contacted with Cy3-conjugated anti-GFAP. FIG. 7B shows phase-contrast photographs (top panels) of O4(+)O1(−) cells that had dedifferentiated from O4(+)O1(+) precursor cells, and fluorescent images of the cells contacted with Cy3-conjugated anti-O4 antibody (left bottom panel) and Cy3-conjugated anti-O1 antibody (right bottom pane). FIG. 7C shows phase-contrast photographs (top panels) of O4(−)O1(−) cells that had dedifferentiated from O4(+)O1(−) precursor cells, and fluorescent images of the cells contacted with Cy3-conjugated anti-O4 antibody (left bottom panel) and Cy3-conjugated anti-O1 antibody (right bottom panel). FIG. 7D shows a phase-contrast photograph of type 2 astrocytes that arose from dedifferentiated O4(−)O1(−) precursor cells, and a fluorescent image of the type 2 astrocytes contacted with Cy3-conjugated anti-GFAP.

FIGS. 8A-8C are photographs of rat oligodendrocyte precursor cells that have differentiated into mature oligodendrocytes and that exhibit myelination around the axons of human dorsal root ganglion (DRG) neuronal axons. FIG. 8A is a phase-contrast photograph of the differentiated cells with DRG; FIG. 8B is a fluorescent image of the same cells immunocytochemically stained with FITC-conjugated anti-neurofilament 200 kD antibody that detects neurons; and FIG. 8C is a fluorescent image of the same cells immunocytochemically stained with Cy3-conjugated anti-O1 antibody to detect oligodendrocytes.

### DETAILED DESCRIPTION OF THE INVENTION

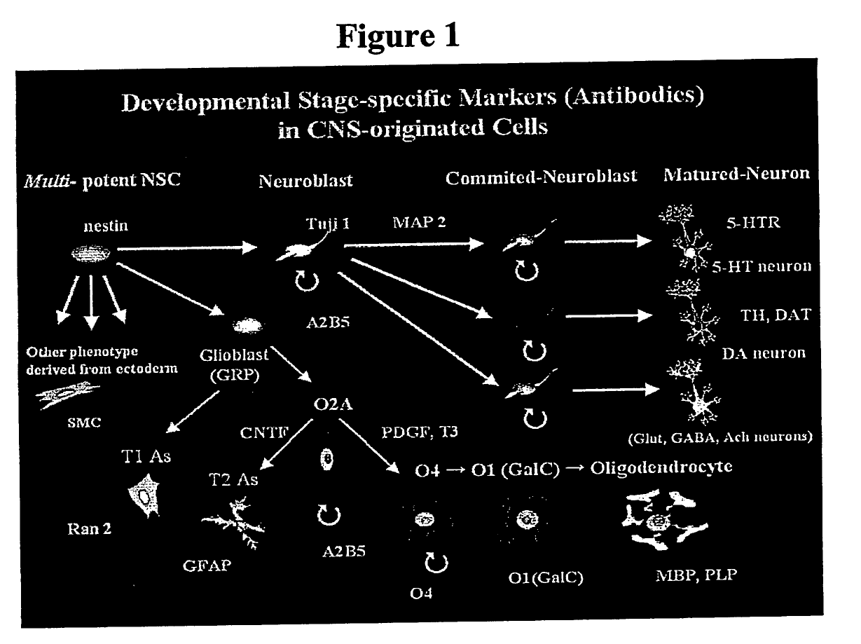

Multipotential neuroepithellal stem cells (NSC) are believed to give rise to all the cells of the central nervous system (CNS). These cells are broadly classified as either neurons or glial cells. Glial cells are further subdivided into astrocytes and oligodendrocytes. The sequential expression of developmental markers, identified by a panel of cell specific antibodies, divide the lineages into distinct phenotypic stages. The cells are also characterized by their proliferative capacities, migratory abilities, and dramatic changes in morphology. FIG. 1 shows a schematic representation of the different cell types characterized by developmental markers and cell morphology. Some of these markers are discussed in more detail below.

Nestin. Nestin is a protein expressed specifically on neuroepithelial stem cells (NSCs) and therefore distinguishes them from other more differentiated cells in the neural tube (Lendahl et al., Cell 60:585-595, 1990). Nestin is also expressed by glial precursors. In culture, high levels of nestin have been observed on proliferating oligodendrocyte progenitors, but the protein becomes down-regulated in differentiated oligodendrocytes (Gallo et al., J. Neurosci. 15:394-406, 1995).

A2B5. The antigen recognized by monoclonal antibody A2B5 (Eisenbarth et al., PNAS 76:4913-4917, 1979) is expressed both on neurons and glial cells in vivo and is used in oligodendrocyte cultures to follow the maturation of oligodendrocyte progenitors. A2B5 antigen becomes downregulated as the cell differentiates into the mature oligodendrocyte.

O4. The monoclonal antibody O4 (Sommer et al., Dev Biol 83:311-327, 1981) marks a specific preoligodendrocyte stage of oligodendrocyte maturation. When a cell binds with the monoclonal antibody O4, the cell is considered O4(+). When a cell does not bind with the monoclonal antibody, the cell is considered O4(−). The role of the O4 marker is discussed in more detail below.

Glycolipids. There are specific glycolipids in oligodendrocytes and myelin, such as galactosylceramides (GalC) (galactocerebrosides) and sulfogalactosylceramides (sulfatides). Galactosylceramides and sulfogalactosylceramides are early markers on oligodendrocyte precursor cells that remain present on the surface of mature oligodendrocytes in culture and in vivo (Pfeiffer et al., Trends Cell Biol 3:191-197, 1993; Raff et al., Brain Res 174:283-308, 1979; Zalc et al., Brain Res 211:341-354, 1981). The main antibody used to identify galactocerebrosides is O1 (Sommer et al., Dev Biol 83:311-327, 1981). Thus, cells expressing GalC are often designated O1(+).

Ganalioside GD3. In vitro, GD3 is highly expressed on oligodendrocyte progenitors and GD3 expression disappears as the cell matures (Hardy et al., Development 111:1061-1080, 1991). In vivo, GD3 is also expressed in other glial cell types, such as immature neuroectodermal cells, subpopulations of neurons and astrocytes, resting ameboid microglia, and reactive microglia.

PSA-NCAM. Expression of the embryonic polysialylated form of neural cell adhesion molecule, PSA-NCAM, is thought to be important for regulation and maintenance of neural structural changes, such as migration, axonal growth, and also for plasticity (Cremer et al., Int. J. Dev. Neuroscl. 18:213-220, 2000). The expression of PSA-NCAM and the absence of GD3 expression together characterize the precursor stage from which oligodendrocyte progenitors arise (Hardy et al., Development 111:1061-1080, 1991; Grinspan et al., J. Neurosci Res 41:540-545,1995).

Myelin basic protein (MBP) and proteolipid protein (PLP). Myelin proteins, which comprise 30% weight of myelin, are specific components of myelin and oligodendrocytes. The major CNS myelin proteins MBP and PLP are low molecular weight proteins and constitute about 80% of the total myelin proteins. Thus, MBP and PLP are specific markers characterizing mature oligodendrocytes.

Glial fibrillary acidic protein (GFAP). Astrocytes contain intermediate filaments, called glial filaments, which are polymers of GFAP and may be readily identified in tissue sections and cultures of CNS by immunohistochemical techniques using anti-GFAP antibodies. Two different types of GFAP+ astrocytes are known to exist: Type 1 astrocytes (T1As) have a fibroblast-like morphology, proliferate in culture, especially in response to epidermal growth factor (EGF), and do not bind to A2B5 antibody; Type 2 astrocytes (T2As) resemble neurons or oligodendrocytes in morphology, divide infrequently in culture, and bind to A2B5 antibody. Type 2 astrocytes appear to develop from A2B5+, GFAP− precursor cells, which rapidly acquire GFAP in culture (Raffet al, J. Neurosci. 3:1289-1300, 1983). The two types of astrocytes do not convert from one type to the other in culture. Id.

A number of features distinguish oligodendrocytes from astrocytes. In particular, oligodendrocytes are smaller in size, have greater density of both the cytoplasm and nucleus, lack intermediate filaments (fibrils) and glycogen in the cytoplasm, and have a large number of microtubules (reviewed in Peters et al., The Fine Structure of the Nervous System: the Neuron and the Supporting Cells. Oxford, UK: Oxford Univ. Press, 1991). An oligodendrocyte may have many cellular extensions, or processes, each of which contacts and repeatedly envelopes a stretch of axon with subsequent condensation of this multispiral membrane-forming myelin. On the same axon, adjacent myelin segments belong to different oligodendrocytes, and every myelin unit terminates near a node of Ranvier (Bunge et al., J. Cell Biol. 12:448-459, 1962; Bunge, Physiol Rev 48:197-210, 1968).

Before their final maturation into myelin-forming cells, oligodendrocytes go through many stages of development defined by the expression of specific cell-surface receptors and response to distinct growth factors. The best defined oligodendrocyte precursor cell is the A2B5(+)O4(−) oligodendrocyte type 2 astrocyte (O-2A) progenitor cell (Noble et al., Glia 15:222-230, 1995; Raff, Science 243:1450-1455, 1989; Miller, Trends Neurosci 19:92-96, 1996; Richardson et al., Semin. Neurosci. 2:445-454, 1990). O-2A progenitor cells are capable of differentiating in vitro into oligodendrocytes and into type 2 astrocytes, but not into type 1 astrocytes. Thus, O-2A progenitor cells are considered to be bipotential. O-2A progenitor cells may be induced to undergo self-renewal, or proliferation, in vitro in the presence of growth factors. For instance, growth in the presence of platelet-derived growth factor (PDGF) is associated with both self-renewal and the generation of oligodendrocytes (Richardson et al., Cell 53:309-319, 1988; Raff et al., Nature 333:562-565, 1988; Noble et al., Nature 333:560-562, 1988), while growth in the presence of both PDGF and basic fibroblast growth factor (bFGF) stimulates continuous self-renewal without differentiation (Bogler et al., PNAS 87:6368-6372, 1990). Notably, when O-2A progenitor cells are cultured in a medium containing bFGF as the only exogenous growth factor added, the O-2A cells undergo premature oligodendrocyte differentiation. Id. O-2A progenitor cells may also be induced to differentiate into type 2 astrocytes when treated with 10% fetal calf serum (Raff et al., Nature 303:390-396, 1983).

Morphologically, O-2A progenitors are generally bipolar (having two major cellular extensions). As O-2A progenitor cells mature, they become multipolar, less motile, but are still proliferative cells which react with the monoclonal antibody O4. This is followed by a transient developmental stage, pre-GalC. These post-O-2A but pre-oligodendrocyte cells have been collectively called A2B5(+)O4(+)O1(−) cells. The onset of terminal differentiation, i.e., the immature oligodendrocyte stage, is identified by the synthesis and transport to the surface of galactosylceramides (GalC), which are reactive to the monoclonal antibody O1. After a characteristic lag of one or two days, mature oligodendrocytes develop with the regulated expression of terminal markers such as the myelin basic protein (MBP) and proteolipid protein (PLP), and the synthesis of the myelin membrane. Although most studies of O-2A progenitors and their more differentiated oligodendrocyte precursor cells have been isolated and studied from rodents, bipolar O-2A cells, multipolar A2B5(+)O4(+) cells, and mature O1(+) oligodendrocytes have also been identified in human fetal brain (Rivkin et al., Ann Neurol 38:92-101, 1995).

More recently, other oligodendrocyte precursor cells have been identified. A tripotential precursor cell termed glial restricted precursor (GRP) has been isolated from the rat spinal cord and has been found to differ from the bipotential O-2A progenitor cells (Rao et al., PNAS 95:3996-4001, 1998; Rao et al., Dev Biol 188:48-63, 1997). Like O-2A progenitor cells, GRPs are A2B5(+) immunoreactive cells and are unable to differentiate into neuronal precursor cells or neurons. Unlike O-2A cells, GRPs are capable of differentiating into oligodendrocytes and both types of astrocytes, type 1 (A2B5(−)GFAP(+)) and type 2 (A2B5(+)GFAP(+)). Moreover, freshly isolated GRPs are unresponsive to PDGF, unlike O-2A cells. The ability of GRP cells to respond to PDGF may be acquired, however, after several days of growth in a medium containing bFGF and PDGF. Morphologically, GRP cells are unipolar or bipolar. It has recently been demonstrated that GRP cells may give rise to O-2A progenitor cells when grown in the presence of PDGF and thyroid hormone (TH) (Gregori et al., J Neuroscl 22:248-256, 2002) and may therefore constitute the earliest identified glial restricted cell.

Another class of glial precursor cells, termed oligospheres, has been identified. Oligospheres are floating cell aggregates which are believed to comprise A2B5 immunoreactive cells (Avellana-Adalid et al., J Neurosci Res 1:558-570, 1996). Oligospheres were originally isolated from rat neonatal brains (Avellana-Adalid et al., J Neurosci Res 1:558-570, 1996) but have subsequently also been isolated from adult rats (Zhang et al., PNAS 96:4089-4094, 1999), canines (Zhang et al., J Neurosci Res 54:181-190, 1998), and embryonic stem cells (Brustle et al., Science 285:754-756, 1999; Mujtaba et al., Dev Biol 214:113-127, 1999; Liu et al., PNAS 97:6126-5131, 2000). Oligospheres divide in culture and may be propagated as floating spheres of undifferentiated cells. Oligospheres may be induced to differentiate by dissociation and attachment. Upon differentiation, oligospheres generate oligodendrocytes and astrocytes. Id. Although the phenotype of these astrocytes have not been characterized, they are presumed to be type 1 astrocytes (Lee et al., Glia 30:105-121, 2000). Thus, this suggests that oligospheres and O-2A progenitor cells are distinct cells.

Looking beyond just neural cells, embryonic stem (ES) cells are the earliest totipotent cells present in the mammal and may also serve as sources of neuroepithellal stem cells, which then may give rise to neurons, oligodendrocytes and astrocytes. Indeed, ES cells transplanted into traumatically injured spinal cord demonstrated that transplanted ES cells survived and differentiated into astrocytes, oligodendrocytes and neurons (McDonald et al., Nature Med. 5:1410-1412, 1999). Others have isolated oligospheres from ES cells and transplanted the oligospheres into the spinal cords of myelin-deficient mutant mice. The ES-derived oligospheres appeared to migrate into the host tissue, produce myelin, and myelinate host axons (Liu et al., PNAS 97:6126-6131, 2000). However, use of embryonic tissue has increasingly become controversial.

Oligodendrocyte development from oligodendrocyte precursors is a highly regulated and still a relatively undefined process involving various environmental factors. Indeed, the ability of multipotent cells to differentiate into glial-restricted cells (glioblasts) and for glioblasts to further differentiate into oligodendrocytes or astrocytes, appears to be mediated by various growth factors and transcription factors. A number of molecules that are considered important in vivo and in vitro are reviewed in Collarini et al., J Cell Sci (Suppl) 15:117-123, 1991; McMorris et al., Brain Pathol 6:313-329, 1996; Lee et al., Glia 30:105-121, 2000; Baumann et al., Physiol Rev 81:871-927, 2001. These include, but are not limited to, platelet-derived growth factor (PDGF), basic FGF (bFGF), insulin-like growth factor I (IGF-I), neurotrophin-3 (NT-3), glial growth factor (GGF or neuregulin), ciliary neurotrophic factor (CNTF), interleukin-6 (IL-6), transforming growth factor (TGF), IL-2, triiodothyronine (T3), retinoic acid (RA), cAMP, growth-regulated oncogene-alpha (GRO-α) and various hormones. Some of these are discussed in more detail below.

Platelet-derived growth factor (PDGF). PDGF has been identified as an important growth factor for both the proliferation of glial cells as well as differentiation into oligodendrocytes and is produced during development by astrocytes and neurons. PDGF likely plays an important role during development, as PDGF-A null mice showed a large reduction, although not a complete absence, of initial oligodendrocyte generation (Fruftiger et al., Development 126:457-467, 1999). However, PDGF receptor alpha (PDGRR-α) has not been observed in the multipotential neuroepithelial cells nor in freshly isolated GRP cells (Rao et al., PNAS 95:3996-4001, 1998). Thus, PDGF may act at later stages of development, rather than at the initial stages when the multipotent nueroepithelial cells generate the more restricted glial precursor.

In vitro, PDGF enhances proliferation and motility of O-2A cells (McKinnon et al., Glia 7:245-254, 1993; Noble et al., Nature 333:560-562, 1988; Raff et al., Nature 333:562-565, 1988; Richardson et al., Cell 53:309-319, 1988) and may serve as a survival factor in vivo for newly generated oligodendrocytes (Barres et al., Cell 70:31-46, 1993). In the absence of PDGF and other environmental signals, progenitor cells stop dividing prematurely and differentiate exclusively into oligodendrocytes (Temple et al., Nature 313:223-225, 1985; Raff et al., Nature 333:562-565, 1988). Even in the presence of PDGF, however, O-2A progenitor cells divide only a limited number of times before an intrinsic timing mechanism in the cells causes them to stop dividing and differentiate into oligodendrocytes (Raff et al., Nature 333:562-565, 1988).

Basic fibroblast growth factor (bFGF or FGF-2). bFGF stimulates proliferation of oligodendrocytes developing in culture (Eccleston et al., Brain Res 210:315-318, 1984; Saneto et al., PNAS 82:3509-3515, 1985; Besnard et al., Neurosci Lett 73:287-292, 1987; Besnard et al., Int J Dev Neurosci 7:401-409, 1989; Behar et al., J Neurosci Res 21:168-180, 1988) and also stimulates oligodendrocyte precursor cell proliferation with the limited proliferating ability in division numbers or period while preventing differentiation into oligodendrocytes (McKinnon et al., Ann N.Y. Acad Sci 638:378-386, 1991; McKinnon et al., Glia 7:245-254, 1993; Qian et al., Neuron 18:81-93, 1997). bFGF acts by upregulating the expression of PDGF-α and thereby increasing the developmental period during which oligodendrocyte progenitors or preoligodendrocytes are able to respond to PDGF (McKinnon et al., Neuron 5:603-614, 1990). Indeed, O-2A cells have been shown to undergo premature differentiation into oligodendrocytes when exposed to bFGF alone, but are not induced to become self-renewing oligodendrocyte precursor cells. O-2A cells are induced to undergo continuous self-renewal when exposed to a combination of bFGF and PDGF (Bogler et al., PNAS 87:6368-6372, 1990). Similarly, tripotential glial precursor cells have been induced to undergo self-renewal in the presence of bFGF and PDGF (Rao et al., PNAS 95:3996-4001, 1998).

Neurotrophin-3 (NT-3). NT-3 is a member of the nerve growth factor family and appears to stimulate proliferation of oligodendrocyte precursor cells only when added with high levels of insulin, PDGF or with other combinations (Barde, Nature 367:371-375, 1994; Barres et al., Neuron 12:935-942, 1994). NT-3 also promotes oligodendrocyte survival in vitro (Barde, Nature 367:371-375, 1994; Barres et al., Cell 70:31-46, 1992).

Cillary neurotrophic factor (CNTF). CNTF is a cytokine that is structurally and functionally similar to the members of the hematopoletic cytokine family. Treatment of gilal progenitor cells with CNTF may induce the appearance of oligodendrocytes (Mayer et al., Development 120:143-153, 1994; Barres et al., Mol Cell Neurosci 8:146-156, 1996, Lachyankar et al., Exp Neurol 144:350-360, 1997). Studies suggest that CNTF requires the presence of PDGF in order to stimulate oligodendrocyte differentiation (Engel et al., Glia 16:16-26, 1996; Fruttiger et al., Development 126:457-467, 1999).

There is also evidence that CNTF may stimulate the production of type 2 astrocytes from O-2A progenitor cells. CNTF, however, is insufficient by itself to induce the development of type 2 astrocytes. Indeed, molecules associated with the extracellular matrix cooperate with CNTF, possibly by mimicking the effect of fetal calf serum (Lillien et al., J Cell Biol 111:635-644, 1990), which has been previously shown to induce type 2 astrocyte differentiation in vitro (Raff et al., Nature 303:390-396, 1983; Temple et al., Nature 313:223-225, 1985).

Triodothyronine (T3). The thyroid hormone, T3, is capable of maintaining the proliferation of oligodendrocyte precursor cells as well as stimulating their differentiation into mature oligodendrocytes (Barres et al., Development 120:1097-1108, 1994; Ibarrola et al., Dev Biol 180:1-21, 1996; Baas et al., Glia 19:324-332, 1997).

Growth-regulated oncogene-alpha (GRO-α). GRO-α is a cytokine that has also been shown to promote proliferation of oligodendrocyte precursor cells (Robinson et al., J Neuroscl 18:10457-10463, 1998).

Retinoic acid and cAMP. cAMP and retinoic acid appear to regulate the differentiation of oligodendrocyte precursor cells (Raible et al., Dev Biol 133:437-446, 1993; Noll et al., Development 120:649-660, 1994).

Glial growth factor (GGF or neuregulin). GGF is another growth factor which has been shown to stimulate proliferation and survival of oligodendrocyte precursor cells (Canoll et al., Neuron 17:229-243, 1996).

Interestingly, some reversion of more differentiated cells into less differentiated cells has been observed, suggesting that certain cells of the CNS possess some plasticity. For example, Canoll et al. observed that by treating mature oligodendrocytes having the phenotype O1(+)MBP(+) with GGF (glial growth factor) decreased the number of cells expressing the mature markers, O1 and MBP (Canoll et al., Mol. Cell Neurosci. 13:79-94, 1999). This phenotypic reversion was also characterized by changes in cell morphology, re-expression of the intermediate filament protein nestin, and the reorganization of the actin cytoskeleton. Basic FGF has also been shown to decrease the number of mature oligodendrocytes in culture (Fressinaud et al., J. Neurosci. Res. 40:285-293, 1995; Hoffman et al., Glia 14:33-42, 1995; Grinspan et al., J. Neurosci. Res. 46:456-464, 1996). However, all showed the reversion of mature oligodendrocytes but not the reversion of oligodendrocyte precursor cells and also failed to achieve a homogeneous population of dedifferentiated cells. Moreover, some evidence suggests that bFGF may trigger apoptotic cell death of mature oligodendrocytes (Muir et al., J. Neurosci. Res. 44:1-11, 1996). Another study demonstrated that type-2 astrocytes could be reverted back to cells having bipolar morphology characteristic of perinatal oligodendrocyte precursor cells (Kondo et al., Science 289:1754-1757, 2000). Gard and Pfeiffer observed that O4(+)O1(−) precursor cells could be transiently reverted to the A2B5(+)O4(−) phenotype in the presence of PDGF. However, the reverted phenotype could not be maintained, as the cells quickly redifferentiated to the O4(+)O1(−) phenotype, and shortly thereafter differentiated into O1(+) oligodendrocytes (Gard et al., Dev. Biol. 159:618-630, 1993).

Despite the work embodied in these various studies, there had been no report of a method for obtaining, maintaining and storing a phenotypically homogeneous population of self-renewing oligodendrocyte precursor cells that are synchronous in their developmental stage. Because of their ability to self-renew, ability to proliferate, ability to terminally differentiate into oligodendrocytes, and phenotypic homogeneity with synchronicity in developmental stage, these cells are useful for the treatment of various CNS disorders and conditions, and for studying the disorders and conditions, both in vitro and in vivo, and the present invention provides these cells.

Before the present invention is further described, it is to be understood that this invention is not limited to particular embodiments described. It is also to be understood that the terminology used herein is for the purpose of describing particular embodiments only, and is not intended to be limiting, since the scope of the present invention will be limited only by the appended claims.

Where a range of values is provided, it is understood that intervening values are encompassed within the invention. The upper and lower limits of these smaller ranges can independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, certain methods and materials are now described. All publications mentioned herein are incorporated herein by reference to disclose and describe the methods and/or materials in connection with which the publications are cited.

It must be noted that as used herein and in the appended claims, the singular forms “a,” “or,” and “the” include plural referents unless the context clearly dictates otherwise.

Further, unless otherwise indicated, all numbers expressing quantities of ingredients, reaction conditions, and so forth, used in the specification and claims are to be understood as being modified in all instances by the term “about.” Accordingly, unless indicated to the contrary, the numerical parameters set forth in the specification and claims are approximations that may vary depending upon the desired properties sought to be obtained by the present invention.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in the respective testing measurements.

As used herein, “phenotypically homogeneous population” and “having a synchronized developmental stage” refer to a population of cells exhibiting substantially the same phenotype and developmental stage. Such a homogeneous population may comprise greater than about 90% of substantially the same cells, or at least about 92%, 94%, 96%, 98%, 99%, 99.9% or 100% of substantially the same cells.

“Oligodendrocyte precursor cell” or “oligodendrocyte progenitor cell” are used herein to describe a cell that has not yet differentiated into a mature oligodendrocyte and that has the potential to differentiate into oligodendrocytes. In one embodiment of the invention, the oligodendrocyte precursor cell may have the potential to differentiate into type 2 astrocytes. In another embodiment, the oligodendrocyte precursor of the invention does not differentiate into type 1 astrocytes. In yet another embodiment, the oligodendrocyte precursor cell may be characterized by the phenotype A2B5(+)O4(−)O1(−); A2B5(+)O4(+)O1(−); or A2B5(+)O4(+)O1(+). A2B5, O4, and O1 refer to surface marker expression of a protein reactive with the antibodies A2B5, O4, and O1, respectively.

Similarity, homogeneity or synchronicity, in developmental stage may be determined by the amount of time a cell takes to produce a more differentiated cell. For example, a homogeneous population of oligodendrocyte precursor cells may be induced to differentiate into a homogeneous population of oligodendrocytes. A heterogeneous population of oligodendrocyte precursor cells may be induced to differentiate into a phenotypically heterogeneous population of oligodendrocytes within a different time period or into another cellular phenotype, such as type 2 astrocyte. If the population comprises developmentally synchronous oligodendrocyte precursor cells, oligodendrocytes or oligodendrocyte precursor cells having further developmental stage may arise within a similar time period. If the population comprises developmentally asynchronous (or unsynchronized) oligodendrocyte precursor cells, oligodendrocytes may arise at varying time periods.

Tissues or cells from which oligodendrocyte precursor cells of the invention may be obtained may be any fetal, juvenile or adult neural tissue, including tissue from the hippocampus, cerebellum, spinal cord, cortex, striatum, basal forebrain, ventral mesencephalon, locus ceruleus, and hypothalamus. The oligodendrocyte precursor cells of the present invention may also be obtained from embryonic stem cells. Moreover, tissues or cells may be obtained from any mammalian species, including, rodents, human, non-human primates, equines, canines, felines, bovines, porcines, ovines, lagomorphs, and the like.

The heterogeneous population of cells comprising oligodendrocyte precursor cells may be obtained from any one of the sources described above and by any of the methods known in the art. For example, Gard et al. describe an immunopanning method by which A2B5(+)O4(+)O1(−) precursor cells in varying developmental stages may be obtained (Gard et al., Neuroprotocols 2:209-218, 1993). The immunopanning method may be modified to obtain A2B5(+)O4(−) precursor cells. McCarthy et al. have also described a cell culture method for obtaining astroglial or oligodenroglial cell cultures (McCarthy et al., J. Cell Biol. 85:890-902, 1980). Other methods known in the art may be used.

The present invention provides a method for obtaining a self-renewing, phenotypically homogeneous population of oligodendrocyte precursor cells having a synchronized developmental stage. The method comprises culturing a heterogeneous population of oligodendrocyte precursor cells having an unsynchronized developmental stage in a medium comprising an effective amount of a fibroblast growth factor (FGF) until a homogeneous population of oligodendrocyte precursor cells having a synchronized developmental stage is obtained. The FGF may a member of the FGF family selected from FGF1, 2, 4, 5, 6, 7, 8b, 9, 10 and 17. Preferred family members include FGF2, 4, 6, 8b, 9 and 17. Most preferred is FGF2, also known as basic FGF (bFGF). An “effective amount of a FGF” refers to the amount of a FGF family member that is effective for inducing a synchronized developmental stage and that supports survival, self renewal, and/or proliferation of a cell.

In one embodiment, the culture medium may comprise FGF at a concentration of between about 0.1 ng/ml and about 40 ng/ml. Preferably, the concentration of FGF is at least about 0.1 ng/ml, 1 ng/ml, 2.5 ng/ml, 5 ng/ml, 10 ng/ml, 15 ng/ml, 25 ng/ml, 30 ng/ml or 40 ng/ml. In an embodiment of the invention, the FGF concentration in the culture medium may be about 0.1 ng/ml to about 40 ng/ml. In another embodiment, the FGF concentration may be about 1 to about 10 ng/ml. In yet another embodiment, the FGF concentration may be about 2.5 to about 7.5 ng/ml. Preferably, the FGF concentration is about 5 ng/ml. Preferably, the FGF used in the culture medium is bFGF. The same concentrations at which bFGF is used may also be used when other FGF family members are used in place of, or in addition to, bFGF.

In an embodiment of the present invention, the culture medium comprises an effective amount of a FGF, in the substantial absence of platelet-derived growth factor (PbGF). In a preferred embodiment of the invention, the culture medium comprises an effective amount of bFGF, in the substantial absence of platelet-derived growth factor (PDGF). As explained above, an “effective amount of FGF” refers to the amount of FGF that is effective for inducing a synchronized developmental stage and that is sufficient to support survival, self renewal, and/or proliferation of a cell.

A “substantial absence of PDGF” refers to the absence of PDGF in the culture medium. Preferably, there is less than about 0.1 ng/ml of PDGF in the culture medium. More preferably, there is less than about 0.01 ng/ml of PDGF in the culture medium. Most preferably, there is less than about 0.001 ng/ml of PDGF in the culture medium. It is understood that PDGF may be endogenously produced by the cultured cells and thus complete absence of PDGF from the culture medium may not be possible. Thus, in an embodiment of the invention, the culture medium may comprise an effective amount of bFGF and a trace amount of PDGF that does not have an effect on survival, self renewal and/or proliferation. Alternatively, the culture medium may comprise an effective amount of bFGF that may stimulate production of endogenous PDGF by the cultured cells.

In one embodiment of the present invention, the method for obtaining a self-renewing, phenotypically homogeneous population of oligodendrocyte precursor cells having a synchronized developmental stage may comprise one or more culturing steps prior to culturing the heterogeneous population of cells comprising oligodendrocyte precursor cells in the medium comprising an effective amount of bFGF in the substantial absence of PDGF. For example, A2B5(+)O4(−) cells obtained by the immunopanning method may be initially cultured in a medium comprising PDGF and bFGF, and any other growth factor. When the cells are switched to medium comprising bFGF in the substantial absence of PDGF, the cells will still generally be heterogeneous in that they are phenotypically and/or developmentally asynchronous.

The self-renewing, phenotypically homogeneous population of oligodendrocyte precursor cells having a synchronized developmental stage obtained according to the method of the invention may possess one or more of the following characteristics. For example, the cells may proliferate, or self-renew, in response to bFGF without observable differentiation and without added PDGF in the medium. The use of the terms “proliferate” and “self-renewing”, with regard to the oligodendrocyte precursor cells, refers to cells that possess the capability of continuous cell division without phenotypic change in the resulting cells. Indeed, as shown in the Examples below, a unique self-renewing, phenotypically homogeneous population of cells having a synchronized developmental stage that is capable of proliferating indefinitely without differentiating, in response to bFGF alone, has been isolated. The cells of the present invention have been continuously culture for more than one year. Thus, as used herein, reference to the ability of cells to proliferate in long-term culture refers to culturing for at least one year.

As another example, because the oligodendrocyte precursor cells obtained are homogeneous, they may be capable of generating a homogeneous population of oligodendrocytes or type 2 astrocytes. The oligodendrocyte precursor cells of the invention may be induced to generate oligodendrocytes by any method known in the art, such as by culturing the cells in serum-free medium without any exogenously added growth factors, or in a medium comprising cilliary neurotrophic factor (CNTF) and/or the thyroid hormone T3 (3,3′,5′-triiodothyronine). When CNTF is used, a preferred concentration range is about 1 ng/ml to about 20 ng/ml. When thyroid hormone T3 is used, a preferred concentration range is about 1 ug/ml to about 30 ug/ml. The oligodendrocyte precursor cells of the invention may be induced to generate type 2 astrocytes by culturing the cells in a medium comprising bone morphogenic protein 2 (BMP-2), BMP-4, or 10% fetal bovine serum. When BMP-2 or BMP-4 is used, a preferred concentration range is about 1 ng/ml to about 20 ng/ml. Other methods of inducing differentiation of oligodendrocyte precursor cells are known in the art.

Also because the oligodendrocyte precursor cells obtained by the methods of the present invention are homogeneous, a population of such cells can be substantially restricted to a single differentiation lineage. That is, all or substantially all of the oligodendrocyte precursor cells in a population can be restricted to develop into oligodendrocytes or type 2 astrocytes. As used herein, “substantially restricted” means greater than about 90%, 91%, 92%, 93%, 94%, 95%, 96%, 97%, 98%, 99% or 100% of the oligodendrocyte precursor cells in a population differentiate into the same mature cell type.

The self-renewing, phenotypically homogeneous population of oligodendrocyte precursor cells having a synchronized developmental stage of the present invention may also be freeze-thawed with high viability and without any change in phenotypic or developmental alterations. As used herein, high viability and a high degree of survival mean greater than about 60%, 70%, 80%, 90%, 95%, 96%, 97%, 98% or 99% viability after freezing and thawing. The cells may be frozen in a culture medium or buffer, with or without bFGF. Upon thawing, the cells maintain their homogeneity as well as their status as oligodendrocyte precursor cells. The medium will also contain a cryoprotectant, such as 5-10% DMSO or glycerol.

The oligodendrocyte precursor cells of the present invention may also be frozen, and maintained in a frozen state, in the culture medium taught herein in the substantial absence of growth factors, such as bFGF. The skilled artisan will understand that other cell freezing buffers known in the art will produce an acceptable level of viability for the cells of the present invention as well

The self-renewing phenotypically homogeneous population of oligodendrocyte precursor cells having a synchronized developmental stage of the invention may also be capable of producing myelin. Myelination may be induced by co-culturing the oligodendrocyte precursor cells of the invention with neurons obtained from the central nervous system, including neurons from the hippocampus, cerebellum, spinal cord, cortex, striatum, basal forebrain, ventral mesencephalon, locus ceruleus, and hypothalamus, or neurons obtained from the peripheral nervous system, including neurons from the dorsal root ganglion (DRG).

The self-renewing, phenotypically homogeneous population of oligodendrocyte precursor cells having a synchronized developmental stage may also be maintained indefinitely in culture in substantially the same phenotypic and developmental state. Thus, the present invention also relates to a method of maintaining a self-renewing, phenotypically homogeneous population of oligodendrocyte precursor cells having a synchronized developmental stage in culture. The method comprises culturing a homogeneous population of oligodendrocyte precursor cells having a synchronized developmental stage in a medium comprising an effective amount of a FGF. The culture medium may comprise a FGF at a concentration of between about 0.1 ng/ml and about 40 ng/ml. Preferably, the concentration of FGF is at least about 0.1 ng/ml, 1 ng/ml, 2.5 ng/ml, 5 ng/ml, 10 ng/ml, 15 ng/ml, 25 ng/ml, 30 ng/ml or 40 ng/ml. In an embodiment of the invention, the FGF concentration in the culture medium may be about 0.1 ng/ml to about 40 ng/ml. In another embodiment, the FGF concentration may be about 1 to about 10 ng/ml. In yet another embodiment, the FGF concentration may be about 2.5 to about 7.5 ng/ml. Preferably, the FGF concentration is about 5 ng/ml. Preferably, the FGF used in the culture medium is bFGF. The same concentrations at which bFGF is used may also be used when other FGF family members are used in place of, or in addition to, bFGF.

The present invention also relates to a method of dedifferentiating an oligodendrocyte precursor cell to a developmentally or phenotypically earlier state. For example, an A2B5(+)O4(+)O1(+) precursor cell may be dedifferentiated into an A2B5(+)O4(+)O1(−) precursor cell, and it may, in turn, be dedifferentiated into an O-2-like cell that has the phenotype A2B5(+)O4(−). The A2B5(+)O4(−) precursor cell, which may be capable of differentiating into oligodendrocytes and type 2 astrocytes, may further be dedifferentiated into a glial-restricted precursor-like cell, which may have the ability to differentiate into oligodendrocytes, type 1 astrocytes, and type 2 astrocytes. The method comprises culturing an oligodendrocyte precursor cell in a medium comprising at least one factor that promotes dedifferentiation. The factor that promotes dedifferentiation may include one or more of bFGF, PDGF, neutrophin-3 (NT-3), or other growth factors.

When A2B5(+)O4(+)O1 (+) precursor cells are dedifferentiated into A2B5(+)O4(+)O1 (−) precursor cells, bFGF alone is used, preferably at a concentration of about 0.1 ng/ml to about 40 ng/ml.

When A2B5(+)O4(+)O1(+) precursor cells are dedifferentiated into A2B5(+)O4(−)O1(−) precursor cells, bFGF is used at a concentration of about 0.1 ng/ml to about 40 ng/ml, preferably with PDGF and NT-3, in the growth medium. When PDGF is included, the concentration of PDGF is preferably about 1 ng/ml to about 50 ng/ml. When NT-3 is included, the concentration of NT-3 is preferably about 1 ng/ml to about 10 ng/ml.

When A2B5(+)O4(+)O1(−) precursor cells are dedifferentiated into A2B5(+)O4(−) precursor cells, PDGF alone may be used, preferably at a concentration of about 1 ng/ml to about 50 ng/ml. Preferably, bFGF and NT-3 are included in the growth medium. When bFGF is included, the concentration of bFGF is preferably about 0.1 ng/ml to about 40 ng/ml. When NT-3 is included, the concentration of NT-3 is preferably about 1 ng/ml to about 10 ng/ml.

The method of the invention is capable of producing a homogeneous population of dedifferentiated oligodendrocyte precursor cells. Thus, a homogeneous population of oligodendrocyte precursor cells having a synchronized developmental stage may be cultured in a medium comprising at least one of the factors above that promotes dedifferentiation. The homogeneous population of dedifferentiated oligodendrocyte precursor cells may be maintained in substantially the same phenotypic and developmental state in the same medium comprising at least one factor that promotes dedifferentiation.

The dedifferentiated oligodendrocyte precursor cell of the invention may or may not possess similar properties as those found in nature. For example, the dedifferentiated oligodendrocyte precursor cell of the invention may be phenotypically similar to the O-2A precursor cell, which has the phenotype A2B5(+)O4(−) and a bipolar morphology. Moreover, the dedifferentiated oligodendrocyte precursor cell may be bipotential like the O-2A precursor cell, in that they are both capable of differentiating into oligodendrocytes and type 2 astrocytes. On the other hand, the dedifferentiated oligodendrocyte precursor cell of the invention may respond differently to growth factors than the O-2A precursor cell. For example, the O-2A precursor cell is generally known to respond to bone morphogenic protein (BMP) 2 or 4 and ciliary neurotrophic factor (CNTF) by generating type 2 astrocytes. The dedifferentiated oligodendrocytes precursor cell of the invention may respond to BMP by generating type 2 astrocytes, but may not respond to CNTF.

The oligodendrocyte precursor cells of the invention further relate to a method of screening for compounds which affect the biological function and differentiation state of oligodendrocyte precursor cells. The method comprises contacting the oligodendrocyte precursor cells of the invention with a test compound, and detecting the change in the oligodendrocyte precursor cell and/or in the culturing medium. The change may be an increase or reduction of any characteristic of the oligodendrocyte precursor cell, for example, myelination, differentiation into oligodendrocytes or astrocytes, surface marker expression, growth characteristics such a proliferation speed, cell migration, viability, surface marker expression, release of proteins, dedifferentiation, or cell morphology. Other characteristics may also be detected as changed.

A test compound may be any chemical, protein, peptide, polypeptide, or nucleic acid (DNA or RNA). The test compound may be naturally-occurring or may be synthesized by methods known in the art. For example, a test compound may be a compound which mimics a neurotransmitter, a hormone or other neuroactive compounds. The test compound may also be an antibody. In an embodiment of the present invention, the method of the present invention is used to screen for compounds which affect myelination.

Agents that promote growth and survival of myelin producing cells may be useful for a variety of therapeutic purposes. Diseases and conditions of the nervous system that result from the deterioration of, or damage to, the myelin sheathing generated by myelin producing cells are numerous. Myelin may be lost as a primary event due to direct damage to the myelin or as a secondary event as a result of damage to axons and neurons. Primary events include neurodegenerative diseases such as multiple sclerosis (MS), human immunodeficiency MS-associated myelopathy, transverse myelopathy/myelitis, progressive multi focal leukoencepholopathy, central pontine myelinolysis and lesions to the myelin sheathing (as described below for secondary events). Secondary events include a great variety of lesions to the axons or neurons caused by physical injury in the brain or spinal cord, ischemia diseases, malignant diseases, infectious diseases (such has HIV, Lyme disease, tuberculosis, syphilis, or herpes), degenerative diseases (such as Parkinson's, Alzheimer's, Huntington's, ALS, optic neuritis, postinfectious encephalomyelitis, adrenoleukodystrophy and adrenomyeloneuropathy), schizophrenia, nutritional diseases/disorders (such as folic acid and Vitamin B12 deficiency, Wemicke disease), systemic diseases (such as diabetes, systemic lupus erthematosis, carcinoma), and toxic substances (such as alcohol, lead, ethidium bromide); and iatrogenic processes such as drug interactions, radiation treatment or neurosurgery.

The oligodendrocyte precursor cells of the invention are safe when administered in vivo and are capable of migrating into the host tissue, producing myelin, and myelinating host axons. Moreover, the oligodendrocytes produced from the oligodendrocyte precursor cells of the invention also are capable of producing myelin and myelinating host axons. Thus, the present invention provides a method of treating a patient, or for the benefit of a patient, which comprises administering to the patient a therapeutically effective amount of the oligodendrocyte precursor cell or oligodendrocyte of the invention. “Therapeutically effective” as used herein, refers to that amount of oligodendrocyte precursor cell that is sufficient to reduce the symptoms of the disorder, or an amount that is sufficient to maintain or increase myelination in the patient. The skilled artisan will understand that therapeutically effective amounts of the oligodendrocyte precursor cells of the present invention will differ based on the condition being treated and the characteristics of the patient.

A patient is hereby defined as any person or non-human animal in need of treatment with oligodendrocyte precursor cells or oligodendrocytes, or to any subject for whom treatment may be beneficial, including humans and non-human animals. Such non-human animals to be treated include all domesticated and feral vertebrates. In an embodiment of the present invention, the oligodendrocyte precursor cells or oligodendrocytes to be administered are obtained from the same species as the species receiving treatment. Examples of mammalian species include rodents, human, non-human primates, equines, canines, felines, bovines, porcines, ovines, lagomorphs, and the like.

The oligodendrocyte precursor cell or oligodendrocyte used in the treatment may also contain a nucleic acid vector or biological vector in an amount sufficient to direct the expression of a desired gene(s) in a patient. The construction and expression of conventional recombinant nucleic acid vectors is well known in the art and includes those techniques contained in Sambrook et al., Molecular Cloning: A Laboratory Manual, Vols 1-3 (2d ed. 1989), Cold Spring Harbor Laboratory Press. Such nucleic acid vectors may be contained in a biological vector such as viruses and bacteria, preferably in a non-pathogenic or attenuated microorganism, including attenuated viruses, bacteria, parasites, and virus-like particles.

The nucleic acid vector or biological vector may be introduced into the cells by an ex vivo gene therapy protocol, which comprises excising cells or tissues from a patient, introducing the nucleic acid vector or biological vector into the excised cells or tissues, and reimplanting the cells or tissues into the patient (see, for example, Knoell et al., Am. J. Health Syst. Pharm. 55:899-904, 1998; Raymon et al., Exp. Neurol. 144:82-91, 1997; Culver et al., Hum. Gene Ther. 1:399-410, 1990; Kasid et al., Proc. Natl. Acad. Sci. U.S.A. 87:473-477, 1990). The nucleic acid vector or biological vector may be introduced into excised cells or tissues by, for example, calcium phosphate-mediated transfection (Wigler et al., Cell 14:725, 1978; Corsaro and Pearson Somatic Cell Genetics 7:603, 1981; Graham and Van der Eb. Virology 52:456, 1973). Other techniques for introducing nucleic acid vectors into host cells, such as electroporation (Neumann et al., EMBO J. 1:841-845, 1982), may also be used.

Administration of cells containing the nucleic acid vector or biological vector may provide the expression of a desired gene(s) that is deficient or non-functional in a patient. Examples of such genes include those coding for receptors that respond to dopamine, GABA, adrenaline, nonadrenaline, serotonin, glutamate, acetylcholine and other neuropeptides, as well as the genes for dopamine, GABA, adrenaline, noradrenaline, acetylcholine, gamma-aminobutyric acid, serotonin, L-DOPA, and other neuropeptides. The cells may also be engineered to produce growth factors, such as nerve growth factor (NGF), bFGF, PDGF, or CNTF. In another embodiment, the nucleic acid vector or biological vector may encode an antisense oligonucleotide. Antisense oligonucleotides are small nucleic acids which are complementary to the “sense” or coding strand of a given gene. They are thus able to stably and specifically hybridize with the RNA transcript of a gene and thereby inhibit RNA translation and therefore the downstream events. Uses of antisense oligonucleotides are known in the art. For example, Holt et al., Mol. Cell Biol. 8:963-973, 1988, have shown that antisense oligonucleotides hybridizing specifically with RNA transcripts of the oncogene c-myc, when added to cultured HL60 leukemic cells, inhibit proliferation and induce differentiation. Similarly, Anfossi et al., Proc. Natl. Acad. Sci. USA 86:3379-3383, 1989, have shown that antisense oligonucleotides specifically hybridizing with RNA transcripts of the c-myb oncogene inhibit proliferation of human myelold leukemia cell lines. Some brain tumors are known to express oncogenes, such as sis, myc, src, and n-myc. Thus, the cells of the invention may be engineered to produce antisense oligonucleotides that target and inhibit sis, myc, src, or n-myc. However, the above list of genes is not intended to be exhaustive. Other genes useful for expression in a patient may be determined by one of ordinary skill in the art.

The cells of the invention may be administered by intracerebral grafting. Grafting may involve direct administration of cells into the central nervous system or the ventricular cavities, or subdural administration onto the surface of a host brain. Specific procedures may include drilling a hole and piercing the dura to permit the needle of a microsyringe to be inserted. Alternatively, cells of the invention may be injected intrathecally into the spinal cord. Such methods for grafting are known to those skilled in the art and are described in, for example, Neural Grafting in the Mammalian CNS, Bjorklund and Stenevi, eds., (1985). Indeed, rat oligodendrocyte precursor cells grown in culture have been engrafted back into animals and have been shown to migrate, engraft, differentiate, and myelinate recipient nerve fibers (Espinosa de los Monteros et al., Dev. Neurosci. 14:98-104, 1992).

The cells of the invention may also be co-administered with other agents, such as growth factors, gangliosides, antibiotics, neurotransmitters, neurohormones, toxins, neurite promoting molecules, antimetabolites, and precursors of these molecules such as the precursor of dopamine, L-DOPA. Other agents may be determined by those of ordinary skill in the art.

The present invention is illustrated by the following Examples, which are not intended to be limiting in any way.

### EXAMPLE 1

### Purification of a Homogeneous Population of Rat Oligodendrocyte Precursor Cells

A2B5(+)O4(−) or A2B5(+)O4(+) cells were first obtained from rat embryonic spinal cord (E14-E19) by the sequential detachment method using Petri dishes or the immunopanning method described in Gard et al., Neuroprotocols 2:209-218, 1993, and in McCarthy et al., J. Cell Biol. 85:890-902, 1980. The cells were then cultured on 0.001% poly-L-omitine (Sigma) pre-coated 10 cm culture dishes (Falcon) at a density of about 20,000-50,000 cells/cm² in a medium A (DMEM/N2 (Gibco); 25 ng/ml PDGF (R&D); 15 ng/ml bFGF (R&D); 5 ng/ml NT-3 (R&D); 0.05% bovine serum (Sigma)). Medium A was exchanged every two days and bFGF was replenished daily.

After approximately one week when the plates became sub-confluent, the cells were trypsinized with medium B (0.125% trypsin; 0.26 mM EDTA; Ca(−) Mg(−) Hank's Buffered Saline Solution (Gibco)) at 37° C. for 20 minutes. The trypsinized cells were repiated in a medium C (DMEM/B27 (Gibco); 10 μM 3,3′,5′-triiodothronine (T3) (Sigma); 10 ng/ml bFGF) for approximately one week.

During this one week incubation in a medium C, the cells began to change from a bipolar morphology (A2B5(+)O4(−)) to multipolar morphology, which is characteristic of A2B5(+)O4(+) cells. When virtually all of the cells acquired a sun-like multipolar morphology, cells were trypsinized with medium B and replated in a medium D (DMEM/B27; 15-30 ng/ml bFGF). The cells were trypsinized and replated approximately every week. During the initial period of subculturing in a medium D, some of the cells gave rise to type 2 astrocytes, suggesting that this initial population of cells was still developmentally heterogeneous. Although proliferating A2B5(+)O4(+) cells were induced even in the next step, these non self-renewing cells differentiate into oligodendrocytes or cells of other phenotypes and died after the limited proliferation number and within a period of one month. After that, some self-renewing A2B5(+)O4(+) oligodendrocyte precursor cells were produced and started to proliferate in medium D. The following subculture was repeated for over one year in a medium D.

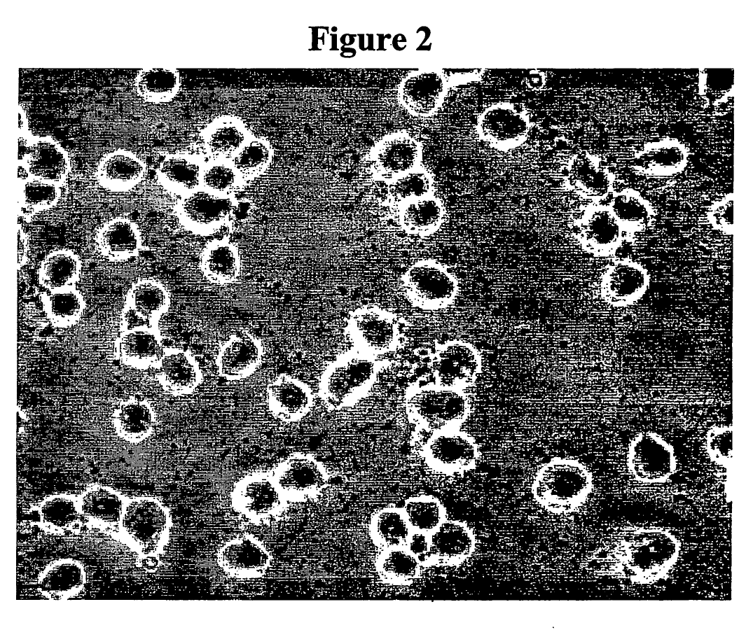

After approximately two months of subculturing in a medium D, type 2 astrocytes no longer appeared and a population containing more than 99.99% (a range of 99.993-99.999% in ten independent studies) self-renewing, phenotypically homogeneous O4(+)O1(−) oligodendrocyte precursor cells was established. Cells were counted by fixing in 4% paraformaldehyde in phosphate saline buffer and staining with anti-O4 antibody (Chemicon) and anti-O1 antibody (Chemicon). Cells were counted in eight random fields per well under a microscope. When the cells were continually maintained in culture, they continued to proliferate and could be maintained for well over a year in a medium D (FIG. 2), without any phenotypic changes or differentiation. Indeed, they were subcultured more than 50 times and maintained for more than 450 days. These cells exhibited unique characteristics, as further demonstrated in the following examples.

The established rat A2B5(+)O4(+)O1(−) oligodendrocyte precursor cell line was deposited under the provisions of the Budapest Treaty with the American Type Culture Collection, 10801 University Blvd., Manassas, Va. 20110-2209 on Jun. 21, 2004, and assigned reference number PTA 6093.

### EXAMPLE 2

### Purification of a Homogeneous Population of Human Oligodendrocyte Precursor Cells

A2B5(+)O4(−) or A2B5(+)O4(+) cells were first obtained from fetal human brain tissue and spinal cord (9-10 weeks) by the immunopanning and/or culture method as described in Example 1. The cells were then cultured on a 0.001% poly-L-omitine and 0.01% laminin pre-coated 10 cm culture dishes (Falcon) at a density of about 20,000-50,000 cells/cm² in a medium A (DMEM/N2 (Gibco); 25 ng/ml PDGF; 5 ng/ml NT-3). Medium A was exchanged every day.

After approximately one week when the plates became sub-confluent, the cells were trypsinized with medium B (0.125% trypsin; 0.26 mM EDTA; Ca(−) Mg(−) Hank's Buffered Saline Solution (Gibco)) at 37° C. for 20 minutes. The trypsinized cells were replated in a medium C (DMEM/B27 (Gibco); 10 μM 3,3′,5′-triiodothronine (T3); 10 ng/ml bFGF) for approximately one month.

During this one-month incubation in a medium C, the cells began to change from a bipolar morphology (A2B5(+)O4(−)) to multipolar morphology, which is characteristic of A2B5(+)O4(+) cells. When virtually all of the cells acquired a sun-like multipolar morphology and proliferating colonies arose in response to bFGF, these colonies of cells were trypsinized with medium B using a microcylinder and were replated in a medium D (DMEM/B27; 15-30 ng/ml bFGF). The cells were trypsinized and replated approximately every week and subculture was repeated indefinitely in a medium D. During the initial period of subculturing in a medium D, some of the cells gave rise to type 2 astrocytes, suggesting that this initial population of cells was still developmentally heterogeneous. Although proliferating A2B5(+)O4(+) cells were induced even in the next step, these non self-renewing cells differentiate into oligodendrocytes or cells of other phenotypes and died after the limited proliferation number and within a period of one month. After that, some self-renewing A2B5(+)O4(+) oligodendrocyte precursor cells were produced and started to proliferate in medium D. The following subculture was repeated for over one year in a medium D.

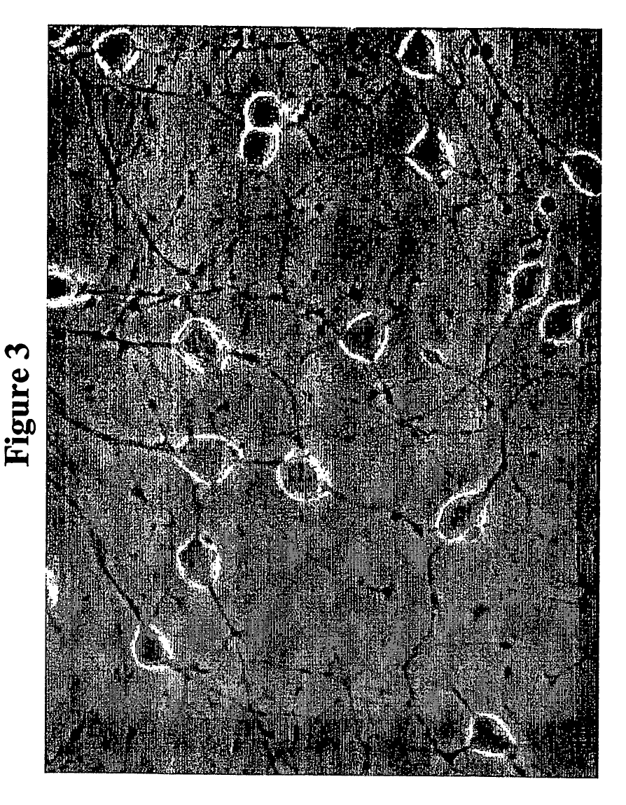

After approximately one month of subculturing in a medium D, type 2 astrocytes no longer appeared and more than a 99.99% homogeneous population of O4(+)O1(−) oligodendrocyte precursor cells was established (FIG. 3). The homogeneous population of human oligodendrocyte precursor cells exhibited similar characteristics as that from rat.

### EXAMPLE 3

### Oligodendrocyte Precursor Cells may be Freeze-Thawed

The rat and human oligodendrocyte precursor cells obtained according to Examples 1 and 2, respectively, were frozen in 5-10% DMSO in DMEM/B27 medium supplemented with or without 15 ng/ml bFGF. When the cells were thawed and cultured in a medium D, the cells were recovered at an average of 90% viability, with a maximum range of 97-99% viability in five independent tests. Moreover, the cells did not show any apparent change in their physical or functional characteristics, such as homogeneity, morphology, proliferation capacity, differentiation ability, and de-differentiation ability. The cells maintained their homogeneity and continued to proliferate without differentiating when cultured in a medium D.

The oligodendrocyte precursor cells obtained according to Examples 1 and 2 may also be frozen, and maintained in a frozen state, in the culture medium taught herein in the substantial absence of growth factors or supplements, such as bFGF or B27 supplement. Such cells also have a high degree of viability upon thawing and culturing (results not shown). The skilled artisan will understand that other cell freezing buffers known in the art will produce an acceptable level of viability for the cells of the present invention as well.

### EXAMPLE 4

### Oligodendrocyte Precursor Cells may be induced into Proliferating O4(+)O1(+) Precursor Cells

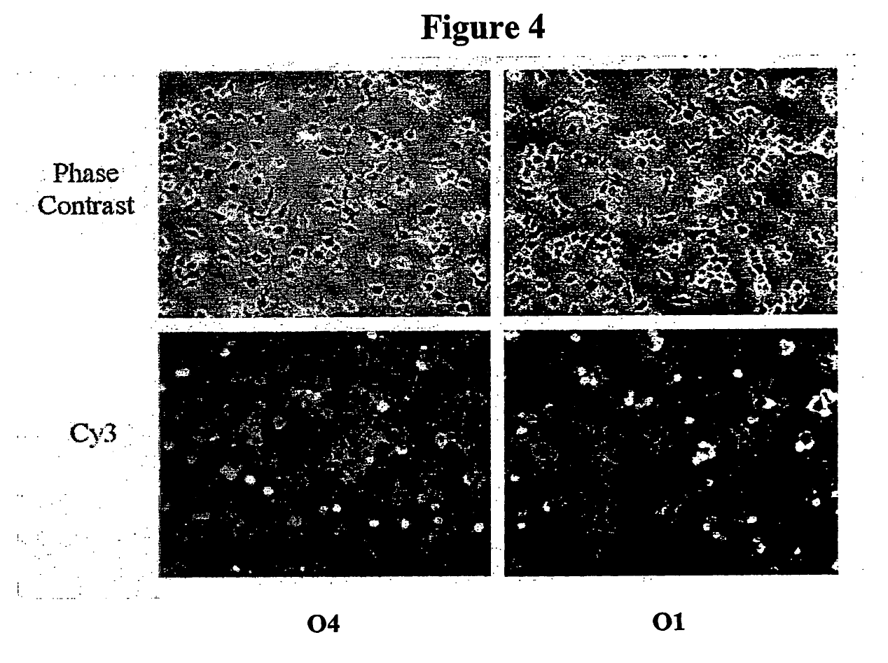

Rat and human oligodendrocyte precursor cells obtained according to Examples 1 and 2, respectively, were induced into O4(+)O1(+) cells at almost 100% efficiency when cultured in DMEM/B27 (Gibco) supplemented with 5 ng/ml CNTF (R&D) and 0.5 ng/ml bFGF (R&D). FIG. 4 shows that these cells are O4(+) by staining with Cy3-conjugated anti-O4 antibody, and O1(+) by staining with Cy3-conjugated anti-O1 antibody. About 98% of the O4(+)O1(+) cells continued to proliferate without fully maturing into oligodendrocytes, as determined by double-staining with anti-BrdU and anti-O1 antibodies 20 hours after 15 μg/ml BrdU incorporation. Thus, the method of the invention may also provide a homogeneous population of proliferating O4(+)O1(+) precursor cells.

### EXAMPLE 5

### Oligodendrocyte Precursor Cells are Capable of Differentiating into Oligodendrocytes

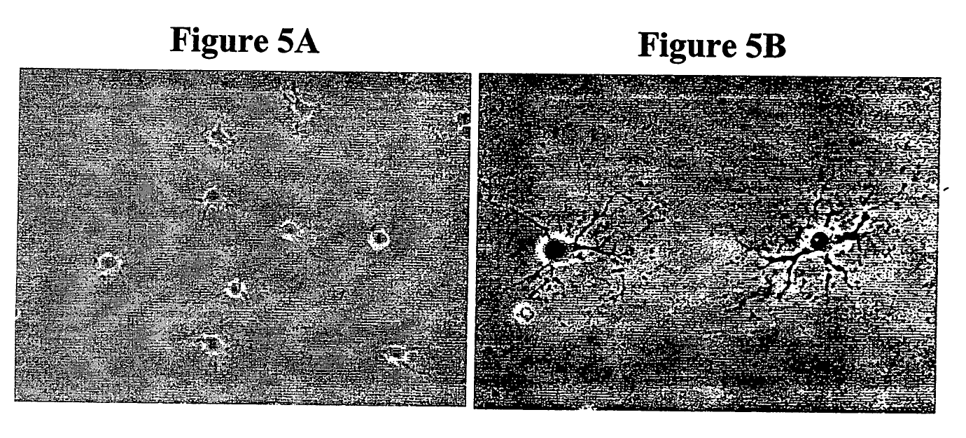

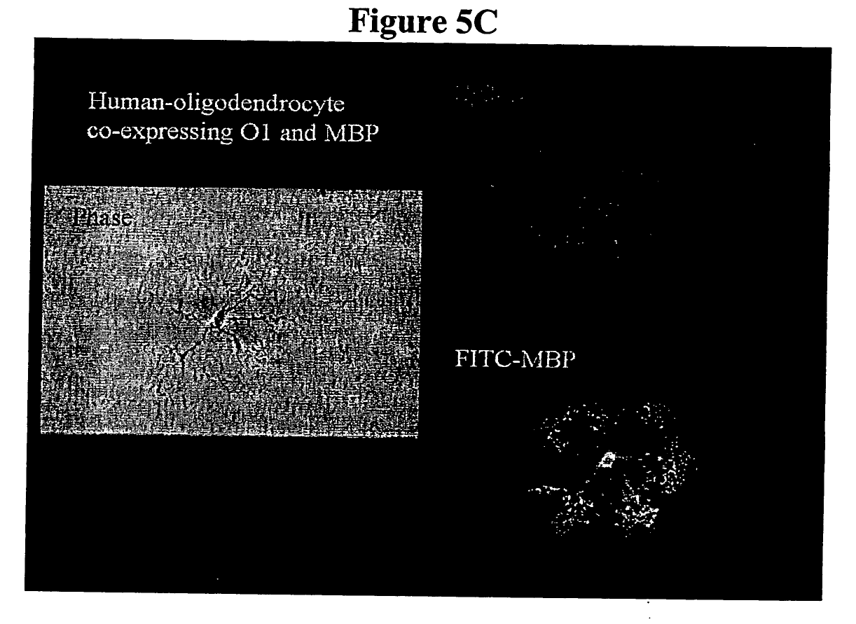

The rat and human oligodendrocyte precursor cells obtained according to Examples 1 and 2, respectively, are also capable of differentiating into oligodendrocytes. The cells were cultured in serum-free conditioned medium (DMEM supplemented with N2) (Gibco). After one week, virtually all of the cells expressed O1 and the myelin basic protein (MBP) (Chemicon), which is a marker expressed on mature oligodendrocytes (FIGS. 5A, 5B, and 5C). Thus, the oligodendrocyte precursor cells obtained were capable of being induced into oligodendrocytes at about 100% efficiency, providing evidence that the rat and human oligodendrocyte precursor cells obtained in Examples 1 and 2, respectively, were all synchronous in their developmental stage.

### EXAMPLE 6

### Oligodendrocyte Precursor Cells are Capable of Differentiating into Astrocytes

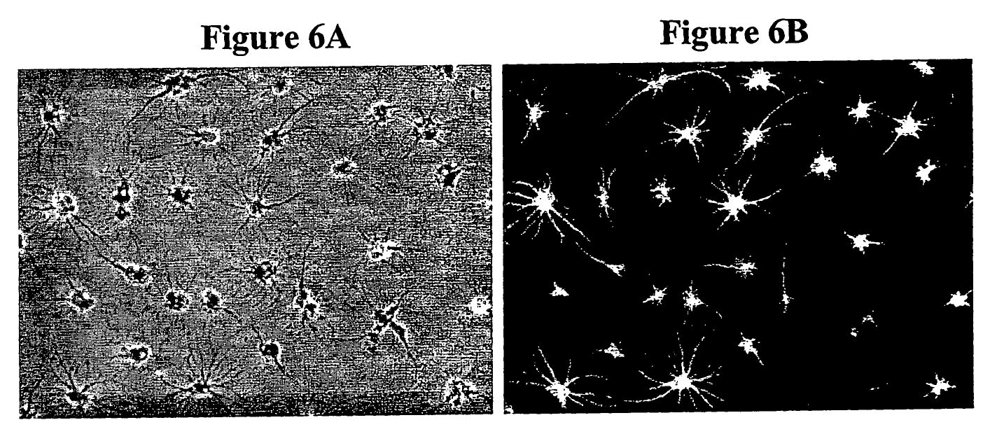

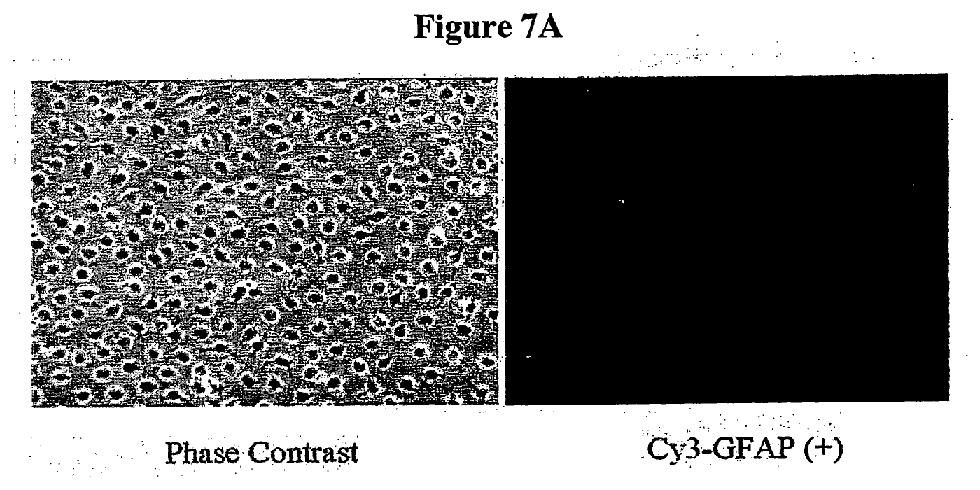

The rat oligodendrocyte precursor cells obtained according to Example 1 are capable of differentiating into type 2 astrocytes. When the cells obtained according to Example 1 were cultured in DMEM/N2 (Gibco) supplemented with 10 ng/ml bone morphogenic protein 2 (BMP-2) or BMP-4 (R&D), virtually all of the cells differentiated into cells expressing the surface marker glial fibrillary acidic protein (GFAP) and A2B5, which together are characteristic of type 2 astrocytes (FIGS. 6A and 6B). This is further evidence that the oligodendrocyte precursor cells obtained in Example 1 were all synchronous in their developmental stage.

### EXAMPLE 7

### Oligodendrocyte Precursor Cells are Capable of Dedifferentiating

The inventor has surprisingly found that the O4(+)O1(+) precursor cells obtained according to Example 4 may be induced to dedifferentiate into O-2A-like cells (O4(−)O1(−)) having bipolar morphology. These dedifferentiated cells were bipotent and capable of re-differentiating into both oligodendrocytes and type 2 astrocytes (T2As).

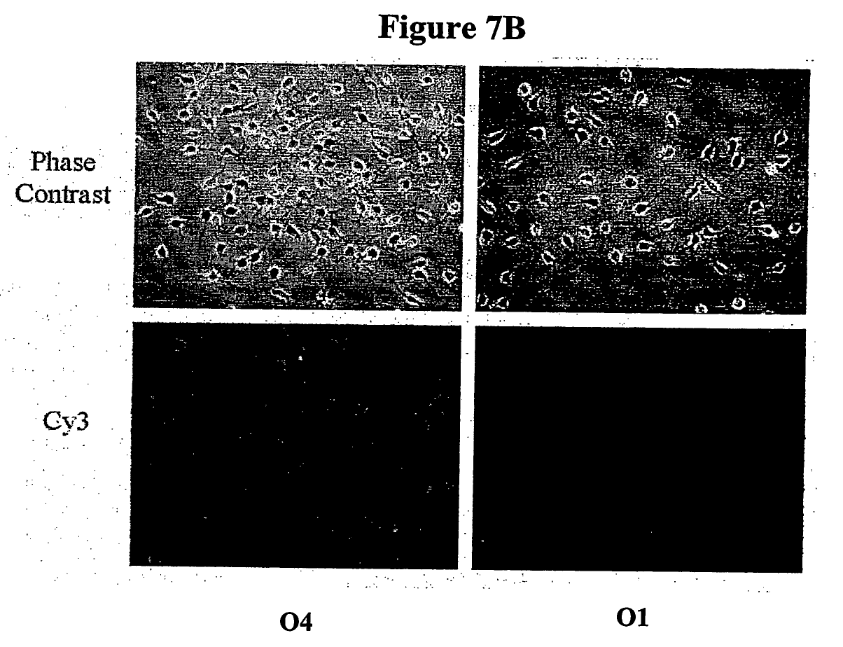

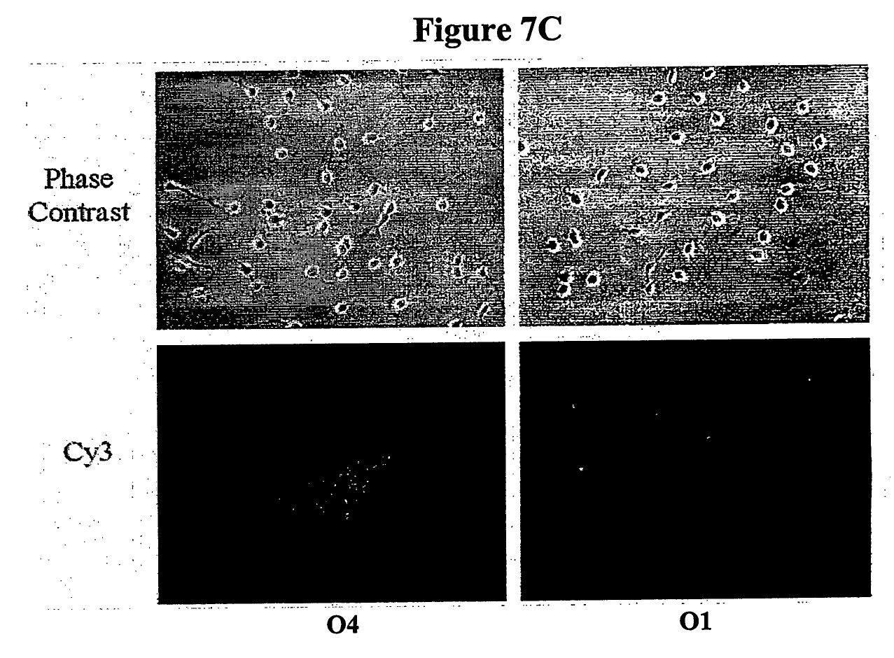

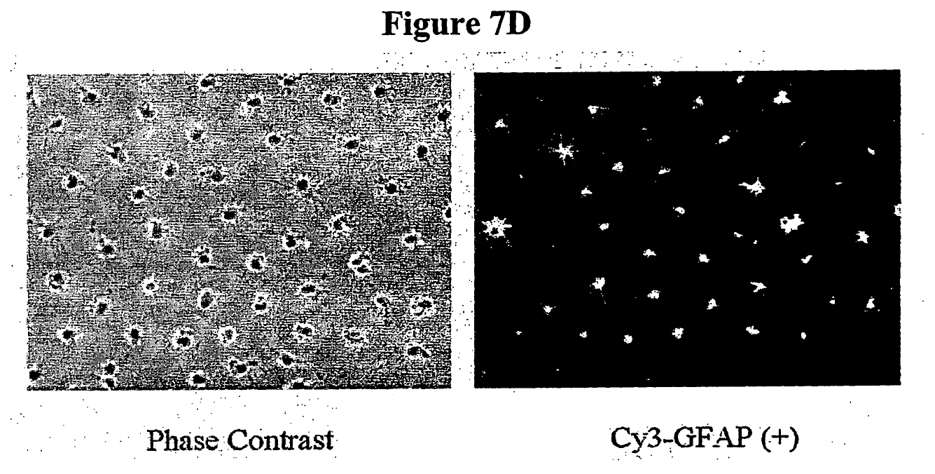

The O4(+)O1(+) precursor cells obtained according to Example 4 were initially cultured in DMEM/N2 supplemented 20 ng/ml BMP-2 or BMP-4 for two weeks but they did not give any GFAP(+)-astrocytes as shown by the lack of staining by a Cy3-conjugated anti-GFAP antibody (Sigma) (FIG. 7A). The cells were then trypsinized and subcultured in DMEM/N2 supplemented with 15 ng/ml bFGF. Virtually all of the cells reverted to O4(+)O1(−) cells after one week as evidenced by the staining of the cells with a Cy3-conjugated anti-O4 antibody but lack of staining with a Cy3-conjugated anti-O1 antibody (FIG. 7B). The medium was then changed to DMEM/N2 supplemented with 25 ng/ml PDGF, 15 ng/ml bFGF, and 5 ng/ml NT3 and cultured and in about one week, virtually all of the cells had further reverted to O4(−)O1(−) cells having bipolar morphology (FIG. 7C). To confirm that the O4(−)O1(−) cells still possessed the bipotential capacity to re-differentiate, the O4(−)O1(−) cells were placed under conditions that would normally give rise to oligodendrocytes or type 2 astrocytes. When the O4(−)O1(−) cells were cultured in serum-free conditioned medium (DMEM supplemented with N2) according to Example 5, almost 100% of the cells differentiated into mature oligodendrocytes that expressed MBP. When the O4(−)O1(−) cells were cultured in a medium containing 10 ng/ml bone morphogenic protein 2 (BMP-2), 10 ng/ml BMP-4, or 10% fetal bovine serum (FBS), the O4(−)O1(−) cells gave rise to type 2 astrocytes at almost 100% efficiency as evidenced by staining with Cy3-conjugated anti-GFAP (FIG. 7D). This is in contrast to O4(+)O1(+) precursor cells, which are non-responsive to BMP and differentiate only into oligodendrocytes. Thus, the dedifferentiated O4(−)O1(−) cells were like O-2A cells in that they were bipotent, capable of giving rise to oligodendrocytes and type 2 astrocytes.

However, the dedifferentiated O4(−)O1(−) cells were distinct from O-2A cells because not only did they lack the O4 surface marker, they also responded differently to at least one environmental factor. For example, O-2A cells are known to respond to CNTF and differentiate into type 2 astrocytes. In contrast, the dedifferentiated O4(−)O1(−) cells of the invention were unresponsive to CNTF. Thus, the O4(−)O1(−) cells obtained may be a newly characterized population of oligodendrocyte precursor cells distinct from the O-2A precursor cell.

### EXAMPLE 8

### Oligodendrocytes are Capable of Myelination

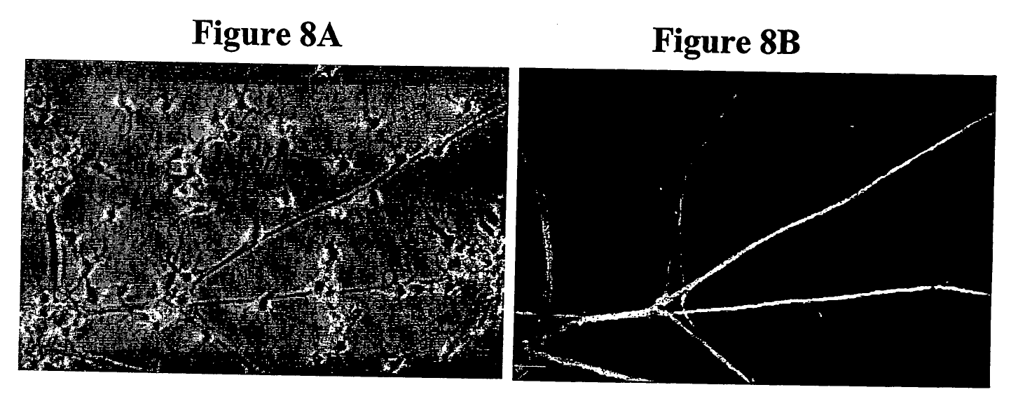

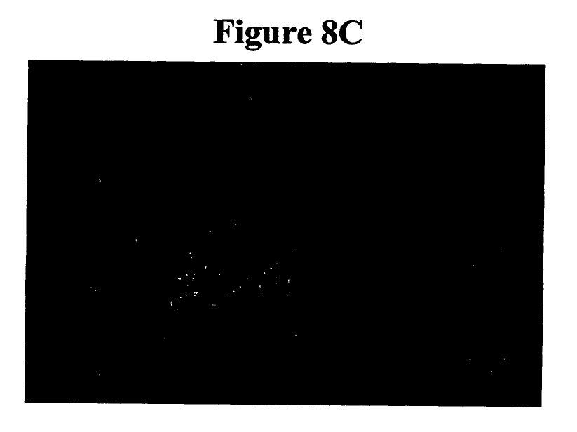

Human DRG neurons (9-10 weeks) were isolated and cultured in DMEM/B27 supplemented with 10 ng/ml CNTF and 10 ng/ml NGF for one week. The oligodendrocyte precursor cells obtained according to Example 1 were added at a neuron:oligodendrocyte precursor cell ratio of 1:2 and co-cultured for more than two weeks. The co-cultured cells were fixed with 4% paraformaldehyde in phosphate saline buffer and then double-stained with a Cy3-conjugated anti-O1 antibody to detect myelin and a FITC-conjugated anti-neurofilament 200 kD antibody (Sigma) to detect axons. More than 99% of the oligodendrocyte precursor cells differentiated into mature oligodendrocytes and some myelinated around the axons of human DRG neurons (FIGS. 8A-8C). In contrast, a control DRG culture that had not been co-cultured with any oligodendrocyte precursor cell did not give rise to any O1(+) oligodendrocytes, nor did the DRGs become myelinated (results not shown).

The specification is most thoroughly understood in light of the teachings of the references cited within the specification, all of which are hereby incorporated by reference in their entirety. The embodiments within the specification provide an illustration of embodiments of the invention and should not be construed to limit the scope of the invention. The skilled artisan recognizes that many other embodiments are encompassed by the claimed invention and that it is intended that the specification and examples be considered as exemplary only, with the true scope and spirit of the invention being indicated by the following claims.

We claim:

1. A method of obtaining a self-renewing, phenotypically homogenous population of oligodendrocyte precursor cells having a synchronized developmental stage, said method comprising culturing a heterogeneous population of oligodendrocyte precursor cells having a unsynchronized developmental stage in a medium comprising an amount of a fibroblast growth factor (FGF) effective for inducing a synchronized developmental stage, wherein said culturing is carried out in substantial absence of platelet-derived growth factor (PDGF), thereby obtaining said self-renewing, phenotypically homogeneous population of oligodendrocyte precursor cells having a synchronized developmental stage.

2. The method of claim 1, wherein said self-renewing, homogeneous population of oligodendrocyte precursor cells comprises oligodendrocyte precursor cells that are culturable for at least about one year without phenotypic change in a medium comprising an amount of a fibroblast growth factor (FGF) effective to prevent said phenotypic change, and in the substantial absence of PDGF.

3. The method of claim 1, wherein the cells of said heterogeneous population of oligodendrocyte precursor cells have different developmental rates in the differentiation process, produce cells of different phenotypes upon differentiation, or respond differently to environmental culturing conditions.

4. The method of claim 1, wherein the cells of said homogeneous population of oligodendrocyte precursor cells have the same developmental rate in the differentiation process, produce cells of the same phenotype upon differentiation, or respond in the same manner to environmental culturing conditions.

5. The method of claim 1, wherein the unsynchronized population of oligodendrocyte precursor cells comprises at least two populations of oligodendrocyte precursor cells, each having a different developmental stage, wherein said developmental stage is selected from the group consisting of the A2B5(+)O4(−)O1(−) developmental stage, the A2B5(+)O4(+)O1(−) developmental stage and the A2B5(+)O4(+)O1(+) developmental stage.

6. The method of claim 1, wherein the synchronized population of oligodendrocyte precursor cells is a population of oligodendrocyte precursor cells having a A2B5(+)O4(−)O1(−) developmental stage, a A2B5(+)O4(+)O1(−) developmental stage or a A2B5(+)O4(+)O1(+) developmental stage.

7. The method of claim 1, wherein said homogeneous population of oligodendrocyte precursor cells are restricted to the oligodendrocyte lineage.

8. The method of claim 1, wherein the FGF is selected from the group consisting of FGF1, FGF2 (basic FGF or bFGF), FGF4, FGF6, FGF8b, FGF9 and FGF17.

9. The method of claim 1, wherein the FGF is bFGF.

10. The method of claim 1 or 9, wherein the amount of FGF is about 0.1 ng/ml to about 40 ng/ml.

11. The method of claim 10, wherein the amount of FGF is about 1 ng/ml to about 10 ng/ml.

12. The method of claim 11, wherein the amount of FGF is about 5 ng/ml.

13. The method of claim 1, wherein the substantial absence of PDGF is an amount of PDGF less than 0.1 ng/ml.

14. The method of claim 1, wherein the substantial absence of PDGF is an amount of PDGF less than 0.01 ng/ml.

15. The method of claim 1, wherein the substantial absence of PDGF is an amount of PDGF less than 0.001 ng/ml.

16. The method of claim 1, wherein the heterogeneous population of oligodendrocyte precursor cells is obtained from a mammal selected from the group consisting of a rodent, a human, a non-human primate, an equine, a canine, a feline, a bovine, a porcine, an ovine and a lagomorph.

17. The method of claim 1, wherein the heterogeneous population of oligodendrocyte precursor cells is isolated from a member selected from the group consisting of hippocampus, cerebellum, spinal cord, cortex, striatum, basal forebrain, ventral mesencephalon, locus ceruleus, and hypothalamus.

18. The method of claim 1, wherein said homogeneous population of oligodendrocytes precursor cells has at least one characteristic selected from:
(i) ability to generate a homogeneous population of oligodendrocytes;
(ii) ability to generate a homogeneous population of type 2 astrocytes;
(iii) ability to dedifferentiate; and
(iv) lack of ability to differentiate into type 1 astrocytes.

19. A self-renewing, phenotypically homogenous population of oligodendrocyte precursor cells having a synchronized developmental stage obtained by the method of claim 1.

20. A self-renewing, phenotypically homogenous population of oligodendrocyte precursor cells having a synchronized developmental stage obtained by the method of claim 1, wherein said homogeneous population of oligodendrocyte precursor cells are restricted to the oligodendrocyte lineage.

21. A method of obtaining a homogeneous population of differentiated oligodendrocytes comprising culturing a self-renewing, phenotypically homogeneous population of oligodendrocyte precursor cells having a synchronized developmental stage in a medium selected from the group consisting of (a) a medium lacking any mitogens, (b) a medium comprising an amount of cilliary neurotrophic factor (CNTF) sufficient to induce differentiation of said oligodendrocyte precursor cells, (c) a medium comprising an amount of a 3,3′,5′-triiodothyronine (T3) sufficient to induce differentiation of said oligodendrocyte precursor cells, and (d) a medium comprising an amount of cilliary neurotrophic factor (CNTF) and an amount of 3,3′,5′-triiodothyronine (T3) sufficient to induce differentiation of said oligodendrocyte precursor cells.

22. The method of claim 21, wherein the amount of CNTF is about 1 ng/ml to about 20 ng/ml.

23. The method of claim 21, wherein the amount of T3 is about 1 μg/ml to about 30 μg/ml.

24. A method of maintaining an undifferentiated population of oligodendrocyte precursor cells in culture comprising: culturing an undifferentiated population of oligodendrocyte precursor cells in a medium comprising an amount of a fibroblast growth factor (FGF) effective for maintaining an undifferentiated developmental stage, wherein said culturing is carried out in substantial absence of platelet-derived growth factor (PDGF), thereby maintaining an undifferentiated population of oligodendrocyte precursor cells in culture.

25. The method of claim 24, wherein the FGF is selected from the group consisting of FGF1, FGF2 (basic FGF or bFGF), FGF4, FGF6, FGF8b, FGF9 and FGF17.

26. The method of claim 24, wherein the FGF is bFGF.

27. The method of claim 24 or 26, wherein the amount of FGF is about 0.1 ng/ml to about 40 ng/ml.

28. The method of claim 27, wherein the amount of FGF is about 1 ng/ml to about 10 ng/ml.

29. The method of claim 28, wherein the amount of FGF is about 5 ng/ml.

30. The method of claim 24, wherein the substantial absence of PDGF is an amount of PDGF less than 0.1 ng/ml.

31. The method of claim 24, wherein the substantial absence of PDGF is an amount of PDGF less than 0.01 ng/ml.

32. The method of claim 24, wherein the substantial absence of PDGF is an amount of PDGF less than 0.001 ng/ml.

33. A method of dedifferentiating a self-renewing, phenotypically homogenous population of oligodendrocyte precursor cells having a synchronized developmental stage comprising: culturing a self-renewing, phenotypically homogenous population of oligodendrocyte precursor cells having a synchronized developmental stage in a medium comprising at least one growth factor in an amount effective to promote dedifferentiation.

34. The method of claim 33, wherein said homogenous population of oligodendrocyte precursor cells has an A2B5(+)O4(+)O1(+) developmental stage, and said at least one growth factor is bFGF in an amount of about 0.1 ng/ml to about 40 ng/ml.

35. The method of claim 33, wherein said homogenous population of oligodendrocyte precursor cells has an A2B5(+)O4(+)O1(−) developmental stage, and said at least one growth factor is PDGF in an amount of about 1 ng/ml to about 50 ng/ml.

36. The method of claim 34, wherein said medium further comprises PDGF in an amount of about 1 ng/ml to about 50 ng/ml and neutrophin-3 in an amount of about 1 ng/ml to about 10 ng/ml.

37. The method of claim 35, wherein said medium further comprises bFGF in an amount of about 0.1 ng/ml to about 40 ng/ml and neutrophin-3 in an amount of about 1 ng/ml to about 10 ng/ml.

38. The method of claim 33, wherein the homogeneous population of oligodendrocyte precursor cells is a homogeneous population of oligodendrocyte precursor cells having an A2B5(+)O4(−)O1(−) developmental stage, an A2B5(+)O4(+)O1(−) developmental stage or an A2B5(+)O4(+)O1(+) developmental stage.

39. The method of claim 33, wherein the homogeneous population of oligodendrocyte precursor cells dedifferentiate into a homogeneous population of oligodendrocyte precursor cells having an A2B5(+)O4(−)O1(−) developmental stage or an A2B5(+)O4(+)O1(−) developmental stage.

40. The method of claim 33, wherein the dedifferentiated oligodendrocyte precursor cells can differentiate into oligodendrocytes or into type 2 astrocytes.

41. The method of claim 33, further comprising transferring said oligodendrocyte precursor cells at least once during said culturing step by cell dissociation using a digestive reagent.

42. A population of dedifferentiated oligodendrocyte precursor cells obtained by the dedifferentiation method of claim 33.

43. A self-renewing, phenotypically homogeneous population of oligodendrocyte precursor cells having a synchronized developmental stage that differentiates into type 2 astrocytes in response to treatment with bone morphogenic protein 2 (BMP-2) or BMP-4 in an amount effective to induce differentiation into type 2 astrocytes but not in response to treatment with any amount of ciliary neurotrophic factor (CNTF).

44. A dedifferentiated, homogeneous population of oligodendrocyte precursor cells that differentiates into type 2 astrocytes in response to treatment with bone morphogenic protein 2 (BMP-2) or BMP-4 in an amount effective to induce differentiation into type 2 astrocytes but not in response to treatment with any amount of ciliary neurotrophic factor (CNTF).

45. The method of claim 43 or 44, wherein the amount of BMP-2 and BMP-4 is about 1 ng/ml to about 20 ng/ml.

46. A method of screening for compounds that affect a biological function of a homogeneous population of oligodendrocyte precursor cells, a population of oligodendrocytes or a population of type 2 astrocytes, comprising:
(a) contacting a self-renewing, phenotypically homogeneous population of oligodendrocyte precursor cells having a synchronized developmental stage obtained by the method of claim 1 or a population of oligodendrocytes differentiated from said homogeneous population obtained by the method of claim 1 or a population of type 2 astrocyte differentiated from said homogeneous population obtained by the method of claim 1 with a test compound; and
(b) detecting a change in a biological function of the oligodendrocyte precursor cells, the oligodendrocytes or the type 2 astrocytes.

47. The method of claim 46, wherein said homogeneous population of oligodendrocyte precursor cells is a population selected from the group consisting of oligodendrocyte precursor cells of the A2B5(+)O4(−)O1(−) developmental stage, oligodendrocyte precursor cells of the A2B5(+)O4(+)O1(−) developmental stage and oligodendrocyte precursor cells of the A2B5(+)O4(+)O1(+) developmental stage.

48. The method of claim 46, wherein the change is an increase or reduction in at least one of the characteristics selected from the group consisting of myelination, differentiation Into oligodendrocytes or type 2 astrocytes, proliferation speed, cell migration, viability, gene expression, protein expression, protein levels In the culturing medium, dedifferentiation, growth characteristics, and cell morphology.

49. A method of treating a patient with a disease or condition affecting the central nervous system, comprising administering to the patient a therapeutically effective amount of a self-renewing, phenotypically homogeneous population of oligodendrocyte precursor cells having a synchronized developmental stage obtained by the method of claim 1.

50. The method of claim 49, wherein said oligodendrocyte precursor cells are differentiated or dedifferentiated prior to administration to said patient.

51. The method of claim 49, wherein said disease or condition is a demyelinating disease or a neurodegenerative disease.

52. The method of claim 51, wherein said demyelinating disease is selected from the group consisting of spinal cord injury (SCI), multiple sclerosis (MS), human Immunodeficiency MS-associated myelopathy, transverse myelopathy/myelitis, progressive multi focal leukoencepholopathy, and central pontine myelinolysis lesions to the myelin sheathing.

53. The method of claim 51, wherein said neurodegenerative disease is selected from the group consisting of Alzheimer disease, senile dementia of Alzheimer type (SDAT), Parkinson disease, Huntington disease, ischemia, blindness, and a neurodegenerative disease due to myelinated neuron injury.

54. A substantially pure culture of rat A2B5(+)O4(+)O1(−) self-renewing oligodendrocyte precursor cells having ATCC deposit number PTA 6093.

55. A substantially pure, self-renewing homogenous population of oligodendrocyte precursor cells having a synchronized developmental stage.

56. The oligodendrocyte precursor cells of claim 55, wherein said synchronized developmental stage is A2B5(+)O4(−)O1(−).

57. The oligodendrocyte precursor cells of claim 55, wherein said synchronized developmental stage is A2B5(+)O4(+)O1(−).

58. The oligodendrocyte precursor cells of claim 55, wherein said synchronized developmental stage is A2B5(+)O4(+)O1(+).

59. The oligodendrocyte precursor cells of claim 55, wherein said synchronized developmental stage is bFGF dependent.

60. An admixture of a substantially pure, self-renewing homogenous population of oligodendrocyte precursor cells consisting of at least two different synchronized developmental stages, wherein said developmental stage is selected from the group consisting of the A2B5(+)O4(−)O1(−) developmental stage, the A2B5(+)O4(+)O1(−) developmental stage, and the A2B5(+)O4(+)O1(+) developmental stage.
